(19) United States
(12) Patent Application Publication
(10) Pub. No.: US 2009/0209552 A1
(43) Pub. Date: Aug. 20, 2009

(54) Organic Compounds

(75) Inventors: David Andrew Sandham, West Sussex (GB); Catherine Leblanc, West Sussex (GB); Claire Adcock, West Sussex (GB); Kamlesh Jagdis Bala, West Sussex (GB); Maude Nadine Pierrette Pipet, West Sussex (GB)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/304,576

(22) PCT Filed: Jun. 11, 2007
(86) PCT No.: PCT/EP07/05129

§ 371 Date: Dec. 12, 2008

(30) Foreign Application Priority Data
Jun. 13, 2006 (GB) 0611695.8

(51) Int. Cl. A61K 31/496; C07D 401/14

(52) U.S. Cl. 514/253.09; 544/364

(57) ABSTRACT

There are provided according to the invention compounds of formula (I)

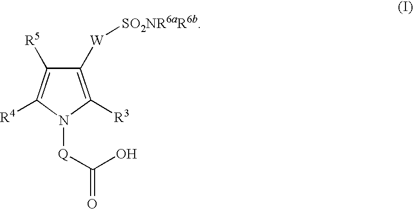

in free or salt form, wherein R³, R⁴, R⁵, R^{6a}, R^{6b}, Q and W are as described in the specification, process for preparing them, and their use as pharmaceuticals.

The present invention relates to organic compounds, their preparation and their use as pharmaceuticals.

In a first aspect, the present invention provides compounds of formula (I)

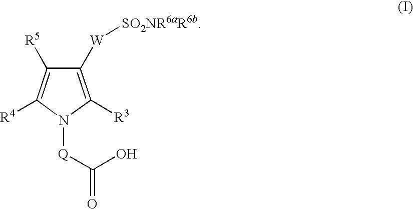

in free or pharmaceutically acceptable salt form, wherein
Q is

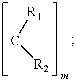

R¹ and R² are, independently, H, halogen, C₁-C₈-alkyl, or together with the carbon atom to which they are attached, form a divalent C₃-C₈-cycloaliphatic group;
R³ and R⁴ are independently selected from H, C₁-C₈-alkyl optionally substituted by C₃-C₁₅ carbocyclic group, or a C₃-C₁₅ carbocyclic group;
R⁵ is selected from H, halogen, C₁-C₈-alkyl, C₁-C₈-haloalkyl, a C₃-C₁₅-carbocyclic group, nitro, cyano, SO₂R^{5a}, SOR^{5b}, SR^{5c}, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, amino(C₁-C₈-alkyl), C₁-C₈-alkylamino, di(C₁-C₈-alkyl)amino, SO₂NR^{5d}R^{5e}, —C(O)NR^{5f}R^{5g}, a C₆-C₁₅-aromatic carbocyclic group, and a 4- to 10-membered heterocyclic group having one or more heteroatoms selected from the group consisting of oxygen, nitrogen and sulphur;
R^{5a}, R^{5b} and R^{5c} are independently selected from C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl and a 4- to 10-membered heterocyclic group having one or more heteroatoms selected from the group consisting of oxygen, nitrogen and sulphur;
R^{5d}, R^{5e}, R^{5f} and R^{5g} are independently H, C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl, a 4- to 10-membered heterocyclic group having one or more heteroatoms selected from the group consisting of oxygen, nitrogen and sulphur, or together with the nitrogen atom to which they are attached, form a C₄-C₁₀-heterocyclic group;
W is selected from C₃-C₁₅-carbocyclic group optionally substituted by halogen, cyano, C₁-C₈-alkyl, or C₁-C₈-haloalkyl, and 4- to 10-membered heterocycle having one or more heteroatoms selected from the group consisting of oxygen, nitrogen and sulphur optionally substituted by halogen, C₁-C₈-alkyl, or C₁-C₈-haloalkyl;
R^{6a} is H or C₁-C₈-alkyl;
R^{6b} is C₁-C₈-alkyl substituted by C₃-C₁₅-carbocyclic group optionally substituted by halogen, C₁-C₈-alkyl, or hydroxyl, or 4- to 10-membered heterocyclic group optionally substituted by halogen, cyano, oxo, hydroxy, carboxy, nitro, or C₁-C₈-alkyl, or
R^{6a} and R^{6b} together with the nitrogen atom to which they are attached, form a 4- to 10-membered heterocyclic group optionally substituted by 4- to 10-membered heterocyclic group, a C₃-C₁₅ carbocyclic group optionally substituted by halogen, C₁-C₈-alkyl or hydroxy, or a C₁-C₈-alkyl optionally substituted by 4- to 10-membered heterocyclic group, or a C₃-C₁₅ carbocyclic group optionally substituted by halogen, C₁-C₈-alkyl or hydroxy;

where each C₃-C₁₅-carbocyclic group, unless otherwise specified, can be optionally substituted by at least one halo, cyano, amino, nitro, carboxy, C₁-C₈-alkyl, C₁-C₈-haloalkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₀-carbocyclic group and a 4- to 10-membered heterocyclic group having at least one ring heteroatom selected from nitrogen, oxygen and sulphur;

and where each 4- to 10-membered heterocyclic group, unless otherwise specified, can be optionally substituted by at least one halo, cyano, oxo, hydroxy, carboxy, nitro, C₁-C₈ alkyl optionally substituted by 4- to 10-membered heterocyclic group, or a C₃-C₁₅ carbocyclic group optionally substituted by halogen, C₁-C₈-alkyl or hydroxy, C₁-C₈-cyanoalkyl, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;

and where each C₆-C₁₅-aromatic carbocyclic group, unless otherwise specified, can be optionally substituted by at least one halo, cyano, amino, nitro, carboxy, C₁-C₈-alkyl, halo-C₁-C₈-alkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₅-carbocyclic group and a 4- to 10-membered heterocyclic group having at least one ring heteroatom selected from nitrogen, oxygen and sulphur; and

m is an integer selected from 1-3.

### DEFINITIONS

Terms used in the specification have the following meanings:

“Optionally substituted”, as used herein, means the group referred to can be substituted at one or more positions by any one or any combination of the radicals listed thereafter.

“Halogen” or “halo” may be fluorine, chlorine, bromine or iodine; preferably it is bromine or chlorine or fluorine.

“C₁-C₈-Alkyl” denotes straight-chain or branched C₁-C₈-alkyl, which may be, e.g., methyl, ethyl, n-propyl, isopropyl, n-butyl, isobutyl, sec-butyl, tert-butyl, straight- or branched-pentyl, straight- or branched-hexyl, straight- or branched-heptyl or straight- or branched-octyl.

“C₃-C₁₅-Carbocyclic group”, as used herein, denotes a carbocyclic group having 3- to 15-ring carbon atoms, e.g., a monocyclic group, either cycloaliphatic, such as a C₃-C₈-cycloalkyl, e.g., cyclopropyl, cyclobutyl, cyclopentyl, cyclohexyl, cycloheptyl or cyclooctyl; or aromatic, such as phenyl, phenylene, benzenetriyl, naphthyl, naphthylene or naphthalenetriyl; or a bicyclic group, such as bicyclooctyl, bicyclononyl including indanyl and indenyl, and bicyclodecyl including naphthyl. Preferably, the C₃-C₁₅-carbocyclic group is a C₃-C₁₀-carbocyclic group, particularly a C₆-C₁₀-aromatic carbocyclic group, e.g., phenyl, phenylene, benzenetriyl, naphthyl, naphthylene or naphthalenetriyl group.

“C₆-C₁₅-Aromatic carbocyclic group”, as used herein, denotes a divalent aromatic group having 6- to 15-ring carbon atoms, e.g., phenylene, naphthylene or anthrylene.

“Divalent C₃-C₈-cycloaliphatic” denotes cycloalkylene having 3- to 8-ring carbon atoms, e.g., a monocyclic group, such as a cyclopropylene, cyclobutylene, cyclopentylene, cyclohexylene, cycloheptylene or cyclooctylene, any of which can be substituted by one or more, usually one or two, C₁-C₄-alkyl groups; or a bicyclic group, such as bicycloheptylene or bicyclooctylene. Preferably “C₃-C₈-cycloalkylene” is C₃-C₅-Cycloalkylene, e.g., cyclopropylene, cyclobutylene or cyclopentylene.

“C₁-C₈-Alkoxy” denotes straight-chain or branched C₁-C₈-alkoxy which may be, e.g., methoxy, ethoxy, n-propoxy, isopropoxy, n-butoxy, isobutoxy, sec-butoxy, tert-butoxy, straight- or branched-pentoxy, straight- or branched-hexyloxy, straight- or branched-heptyloxy or straight- or branched-octyloxy. Preferably, C₁-C₈-alkoxy is C₁-C₄-alkoxy.

“C₁-C₈-Haloalkyl” and “C₁-C₈-haloalkoxy” denote C₁-C₈-alkyl and C₁-C₈-alkoxy, as hereinbefore defined, substituted by one or more halogen atoms, preferably one, two or three halogen atoms, preferably fluorine, bromine or chlorine atoms. Preferably, C₁-C₈-haloalkyl is C₁-C₄-alkyl substituted by one, two or three fluorine, bromine or chlorine atoms. Preferably, C₁-C₈-haloalkoxy is C₁-C₄-alkoxy substituted by one, two or three fluorine, bromine or chlorine atoms. “C₁-C₈-Hydroxyalkyl” denotes C₁-C₈-alkyl as hereinbefore defined, substituted by at least one hydroxy group.

“C₁-C₈-Cyanoalkyl” denotes C₁-C₈-alkyl, as hereinbefore defined, substituted by at least one cyano group.

“C₁-C₈-Alkylsulfonyl”, as used herein, denotes C₁-C₈-alkyl, as hereinbefore defined, linked to —SO₂—. Preferably, C₁-C₈-alkylsulfonyl is C₁-C₄-alkylsulfonyl.

“C₁-C₈-Haloalkylsulfonyl”, as used herein, denotes C₁-C₈-haloalkyl, as hereinbefore defined, linked to —SO₂—. Preferably, C₁-C₈-haloalkylsulfonyl is C₁-C₄-haloalkylsulfonyl, especially trifluoromethylsulfonyl.

“Amino-C₁-C₈-alkyl” and “amino-C₁-C₈-alkoxy” denote amino attached by a nitrogen atom to C₁-C₈-alkyl, e.g., NH₂(C₁-C₈)—, or to C₁-C₈-alkoxy, e.g., NH₂—(C₁-C₈)—O—, respectively, as hereinbefore defined. Preferably, amino-C₁-C₈-alkyl and amino-C₁-C₈-alkoxy are, respectively, amino-C₁-C₄-alkyl and amino —C₁-C₈-alkoxy.

“C₁-C₈-Alkylamino” and “di(C₁-C₈-alkyl)amino” denote amino substituted respectively by one or two C₁-C₈-alkyl groups, as hereinbefore defined, which may be the same or different. Preferably, C₁-C₈-alkylamino and di(C₁-C₈-alkyl)amino are respectively C₁-C₄-alkylamino and di(C₁-C₄-alkyl)amino.

“C₁-C₈-Alkyl amino-C₁-C₈-alkyl” and “di(C₁-C₈-alkyl)amino C₁-C₈-alkyl” denote C₁-C₈-alkyl, as hereinbefore defined, substituted respectively by C₁-C₈-alkylamino or di(C₁-C₈-alkyl)amino, as hereinbefore defined. Preferably, C₁-C₈-alkylamino-C₁-C₈-alkyl and di(C₁-C₈-alkyl)amino-C₁-C₈-alkyl are, respectively, C₁-C₄-alkylamino-C₁-C₄-alkyl and di(C₁-C₄-alkyl)amino-C₁-C₄-alkyl.

“Amino(hydroxy)-C₁-C₈-alkyl” denotes amino attached by a nitrogen atom to C₁-C₈-alkyl and hydroxy attached by an oxygen atom to the same C₁-C₈-alkyl. Preferably, amino-(hydroxy)-C₁-C₈-alkyl is amino-(hydroxy)-C₂-C₄-alkyl.

“Carboxy-C₁-C₈-alkyl” and “carboxy-C₁-C₈-alkoxy” denote carboxy attached by a carbon atom to C₁-C₈-alkyl or C₁-C₈-alkoxy, respectively, as hereinbefore defined. Preferably, carboxy-C₁-C₈-alkyl and carboxy-C₁-C₈-alkoxy are, respectively, carboxy-C₁-C₄-alkyl and carboxy-C₁-C₄-alkoxy.

“C₁-C₈-Alkylcarbonyl”, “C₁-C₈-alkoxycarbonyl” and “C₁-C₈-haloalkylcarbonyl” denote C₁-C₈-alkyl, C₁-C₈-alkoxy or C₁-C₈-haloalkyl, respectively, as hereinbefore defined, attached by a carbon atom to a carbonyl group. “C₁-C₈-Alkoxycarbonyl” denotes C₁-C₈-alkoxy, as hereinbefore defined, wherein the oxygen of the alkoxy group is attached to the carbonyl carbon. Preferably, C₁-C₈-alkylcarbonyl, C₁-C₈-alkoxycarbonyl and C₁-C₈-haloalkylcarbonyl are, respectively, C₁-C₄-alkylcarbonyl, C₁-C₄-alkoxycarbonyl and C₁-C₄-haloalkylcarbonyl.

“C₁-C₈-Alkylamino” and “di(C₁-C₈-alkyl)amino” denote C₁-C₈-alkyl, as hereinbefore defined, attached by a carbon atom to an amino group. The C₁-C₈-alkyl groups in di(C₁-C₈-alkyl)amino may be the same or different. Preferably, C₁-C₈-alkylamino and di(C₁-C₈-alkyl)amino are, respectively, C₁-C₄-alkylamino and di(C₁-C₄-alkyl)amino.

“C₁-C₈-Alkylaminocarbonyl” and “di(C₁-C₈-alkyl)aminocarbonyl” denote C₁-C₈-alkylamino and di(C₁-C₈-alkyl)amino, respectively, as hereinbefore defined, attached by a nitrogen atom to the carbon atom of a carbonyl group. Preferably, C₁-C₈-alkylaminocarbonyl and di(C₁-C₈-alkyl)-aminocarbonyl are, respectively, C₁-C₄-alkylaminocarbonyl and di(C₁-C₄-alkyl)-aminocarbonyl.

“Four (4)- to 10-membered heterocyclic group containing at least one ring heteroatom selected from the group consisting of nitrogen, oxygen and sulphur”, as used herein, may be monocyclic or bicyclic, e.g., furan, tetrahydrofuran, pyrrole, pyrrolidine, pyrazole, imidazole, triazole, isotriazole, tetrazole, thiadiazole, isothiazole, oxadiazole, pyridine, oxazole, isoxazole, pyrazine, pyridazine, pyrimidine, piperidine, piperazine, morpholine, triazine, oxazine, thiazole, quinoline, isoquinoline, benzothiophene, benzoxazole, benzisoxazole, benzothiazole, benzisothiazole, benzofuran, indole, indazolebenzodioxole or benzimidazole. Preferred heterocyclic groups include piperazine, morpholine, imidazole, isotriazole, pyrazole, pyridine, furan, oxazole, oxadiazole, isoxazole, thiazole, tetrazole benzothiophene, benzoxazole, benzothiazole, benzodioxole and benzofuran.

According to formula (I), Q is suitably —CH₂—.

According to formula (I), R³ and R⁴ are, independently, suitably H, C₁-C₈-alkyl optionally substituted by a C₃-C₁₅ carbocyclic group, or a C₃-C₁₅ carbocyclic group. Preferably R³ and R⁴ are both H.

According to formula (I), R⁵ is suitably cyano.

According to formula (I), W is suitably a C₃-C₁₅ carbocyclic group. The C₃-C₁₅ carbocyclic group is suitably a phenyl ring preferably substituted by at least one substituent, such as halogen (e.g. Cl) or C₁-C₈-haloalkyl (e.g. CF₃).

According to formula (I), R^{6a} is suitably H or C₁-C₈-alkyl (e.g. methyl).

According to formula (I), R^{6b} is suitably C₁-C₈-alkyl substituted by a C₃-C₁₅-carbocyclic group (e.g. phenyl) or a 4- to 10-membered heterocyclic group (e.g. furan) optionally substituted by C₁-C₈-alkyl (e.g. methyl).

Also, according to formula (I), the R^{6a} and R^{6b} of —SO₂NR^{6a}R^{6b}, together with the nitrogen to which they are attached form a 4- to 10-membered heterocyclic group, such as piperidine or piperazine. The 4- to 10-membered heterocyclic group can be substituted by a 4- to 10-membered heterocyclic group, preferably a 5- or 6-membered heterocyclic group, such as pyridine. Also, the 4 to 10-membered heterocyclic group can be substituted by a C₁-C₈-alkyl substituted by a 4- to 10-membered heterocyclic group (e.g. pyridine).

Also, the 4- to 10-membered heterocyclic group formed by R^{6a} and R^{6b} of —SO₂NR^{6a}R^{6b} can be substituted by C₃-C₁₅ carbocyclic group optionally substituted by halogen (e.g. Cl or F). Also, the 4- to 10-membered heterocyclic group can be substituted by C₁-C₈-alkyl optionally substituted by a C₃-C₁₅ carbocyclic group (e.g. phenyl).

According to formula (I) m is suitably 1.

Preferred compounds of formula (I), in free or pharmaceutically acceptable salt form, include those of formula (Ia)

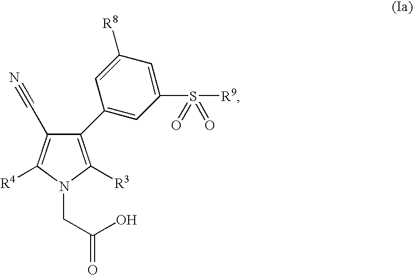

where R³ and R⁴ are as hereinbefore defined, and
R⁸ is selected from halogen and C₁-C₈-haloalkyl;

### R⁹ is NR^{9a}R^{9b};

R^{9a} is H or C₁-C₈-alkyl; and
R^{9b}, C₁-C₈-alkyl substituted by C₃-C₁₅ carbocyclic group or 4- to 10-membered heterocyclic group optionally substituted by C₁-C₈-alkyl, or
R^{9a} and R^{9b} together with the nitrogen atom to which they are attached, form a 4- to 10-membered heterocyclic group optionally substituted by 4- to 10-membered heterocyclic group, a C₃-C₁₅ carbocyclic group optionally substituted by halogen, C₁-C₈-alkyl or hydroxy, or a C₁-C₈-alkyl optionally substituted by 4- to 10-membered heterocyclic group, or a C₃-C₁₅ carbocyclic group optionally substituted by halogen, C₁-C₈-alkyl or hydroxy.

More preferred compounds of formula (I), in free or pharmaceutically acceptable salt form, include those of formula (Ia)

wherein

### R³ and R⁴ are H;

R⁸ is selected from Cl and CF₃; and

R⁹ is selected from

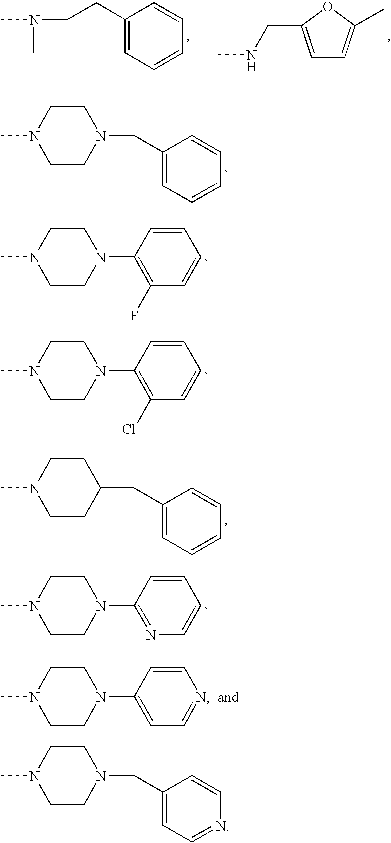

In another embodiment, the present invention provides for the use of a compound of formula (I) in any of the aforementioned embodiments, in free or pharmaceutically acceptable salt form, for the manufacture of a medicament for the treatment of an inflammatory or allergic condition, particularly an inflammatory or obstructive airways disease.

It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments. It is understood by those skilled in the art that only combinations of substituents that are chemically possible are an embodiment of the invention.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps.

Many of the compounds represented by formula (I) are capable of forming acid addition salts, particularly pharmaceutically acceptable acid addition salts. Pharmaceutically acceptable acid addition salts of the compound of formula (I) include those of inorganic acids, e.g., hydrohalic acids, such as hydrochloric acid or hydrobromic acid; nitric acid; sulphuric acid; phosphoric acid; and organic acids, e.g., aliphatic monocarboxylic acids, such as formic acid, acetic acid, diphenylacetic acid, triphenylacetic acid, caprylic acid, dichloroacetic acid, trifluoroacetic acid, hippuric acid, propionic acid and butyric acid; aliphatic hydroxy acids, such as lactic acid, citric acid, gluconic acid, mandelic acid, tartaric acid or malic acid; dicarboxylic acids, such as adipic acid, aspartic acid, fumaric acid, glutamic acid, maleic acid, malonic acid, sebacic acid or succinic acid; aromatic carboxylic acids, such as benzoic acid, p-chlorobenzoic acid, or nicotinic acid; aromatic hydroxy acids, such as o-hydroxybenzoic acid, p-hydroxybenzoic acid, 1-hydroxynaphthalene-2-carboxylic acid or 3-hydroxynaphthalene-2-carboxylic acid; and sulfonic acids, such as ethanesulfonic acid, ethane-1,2-disulfonic acid, 2-hydroxyethanesulfonic acid, methanesulfonic acid, (+)-camphor-10-sulfonic acid, benzenesulfonic acid, naphthalene-2-sulfonic acid, naphthalene-1,5-disulfonic acid or p-toluenesulfonic acid. These salts may be prepared from compounds of formula (I) by known salt-forming procedures.

Compounds of formula (I) contain acidic, e.g., carboxyl, groups, and are also capable of forming salts with bases, in particular, pharmaceutically acceptable bases, such as those well-known in the art; suitable such salts include metal salts, particularly, alkali metal or alkaline earth metal salts, such as sodium, potassium, magnesium, calcium or zinc salts; or salts with ammonia or pharmaceutically acceptable organic amines or heterocyclic bases, such as arginine, benethamine, benzathine, diethanolamine, ethanolamine, 4(2-hydroxyethyl)morpholine, 1-(2-hydroxyethyl)pyrrolidine, N-methyl glucamine, piperazine, triethanolamine or tromethamine. These salts may be prepared from compounds of formula (I) by known salt-forming procedures.

In those compounds where there is an asymmetric carbon atom or an axis of chirality the compounds exist in individual optically active isomeric forms or as mixtures thereof, e.g., as racemic or diastereomeric mixtures. The present invention embraces both individual optically active R and S isomers, as well as mixtures, e.g., racemic or diastereomeric mixtures thereof.

Specific especially preferred compounds of formula (I) include those hereinafter described in the Examples.

Since prodrugs are known to enhance numerous desirable qualities of pharmaceuticals, e.g., solubility, bioavailability, manufacturing, etc., the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers which release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to the parent compound. Prodrugs include compounds of the present invention wherein a carboxy, hydroxy, amino or sulfhydryl-group is bonded to any group that, when the prodrug of the present invention is administered to a mammalian subject, it cleaves to form a free carboxy, free hydroxy, free amino or free sulfhydryl group, respectively. Examples of prodrugs include, but are not limited to, ester derivatives of carboxy functional groups, acetate, formate and benzoate derivatives of alcohol and amine functional groups in the compounds of the present invention.

“Therapeutically effective amount” is intended to include an amount of a compound of the present invention alone or an amount of the combination of compounds claimed or an amount of a compound of the present invention in combination with other active ingredients effective to treat the inflammatory diseases described herein.

As used herein, “treating” or “treatment” cover the treatment of a disease-state in a mammal, particularly in a human, and include:
(a) preventing the disease-state from occurring in a mammal, in particular, when such mammal is predisposed to the disease-state but has not yet been diagnosed as having it;
(b) inhibiting the disease-state, i.e., arresting its development; and/or
(c) relieving the disease-state, i.e., causing regression of the disease state.

### Synthesis

Another embodiment of the present invention provides a process for the preparation of compounds of formula (I), in free or pharmaceutically acceptable salt form, which comprises the steps of:
(i) cleaving an ester group-COOR⁷ in a compound of formula (II)

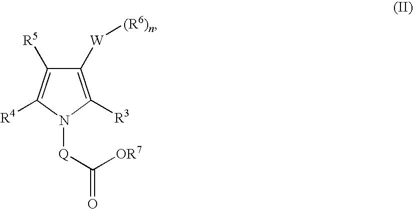

wherein
R⁷ is C₃-C₁₅ carbocyclic group or C₁-C₈-alkyl optionally substituted by a C₃-C₁₅ carbocyclic group; and
everything else as hereinbefore defined; and
(ii) recovering the resultant compound of formula (I), in free or pharmaceutically acceptable salt form.

The process may be carried out using known procedures for ester cleavage or analogously as hereinafter described in the Examples.

Starting materials for the process, and compounds for the preparation of those starting materials, may be novel or known; they may be prepared in accordance with known procedures or analogously, as hereinafter described in the Examples.

Another embodiment of the present invention provides compounds of formula (II)

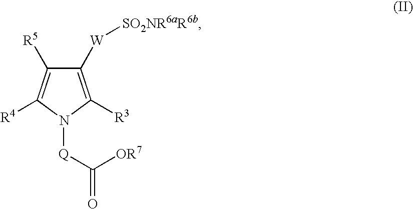

in free or pharmaceutically acceptable salt form,
wherein
Q is

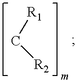

R¹ and R² are, independently, H, halogen, C₁-C₈-alkyl, or together with the carbon atom to which they are attached, form a divalent C₃-C₈-cycloaliphatic group;
R³ and R⁴ are independently selected from H, C₁-C₈-alkyl optionally substituted by C₃-C₁₅ carbocyclic group, or a C₃-C₁₅ carbocyclic group;
R⁵ is selected from H, halogen, C₁-C₈-alkyl, C₁-C₈-haloalkyl, a C₃-C₁₅-carbocyclic group, nitro, cyano, SO₂R^{5a}, SOR^{5b}SR^{5c}, C₁-C₈-alkylcarbonyl, C₁-C₈-alkoxycarbonyl, C C₁-C₈-alkoxy, C₁-C₈-haloalkoxy, carboxy, carboxy-C₁-C₈-alkyl, amino, amino(C₁-C₈-alkyl), C₁-C₈-alkylamino, di(C₁-C₈-alkyl)amino, SO₂NR^{5d}R^{5e}, —C(O)NR^{5f}R^{5g}, a C₆-C₁₅-aromatic carbocyclic group, and a 4- to 10-membered heterocyclic group having one or more heteroatoms selected from the group consisting of oxygen, nitrogen and sulphur;
R^{5a}, R^{5b} and R^{5c} are independently selected from C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl and a 4- to 10-membered heterocyclic group having one or more heteroatoms selected from the group consisting of oxygen, nitrogen and sulphur;
R^{5d}, R^{5e}, R^{5f} and R^{5g} are independently H, C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl, a 4- to 10-membered heterocyclic group having one or more heteroatoms selected from the group consisting of oxygen, nitrogen and sulphur, or together with the nitrogen atom to which they are attached, form a C₄-C₁₀-heterocyclic group;
W is selected from C₃-C₁₅-carbocyclic group optionally substituted by halogen, cyano, C₁-C₈-alkyl, or C₁-C₈-haloalkyl, and 4- to 10-membered heterocycle having one or more heteroatoms selected from the group consisting of oxygen, nitrogen and sulphur optionally substituted by halogen, C₁-C₈-alkyl, or C₁-C₈-haloalkyl;
R^{6a} is H or C₁-C₈-alkyl;
R^{6b} is C₁-C₈-alkyl substituted by C₃-C₁₅-carbocyclic group optionally substituted by halogen, C₁-C₈-alkyl, or hydroxyl, or 4- to 10-membered heterocyclic group optionally substituted by halogen, cyano, oxo, hydroxy, carboxy, nitro, or C₁-C₈-alkyl, or
R^{6a} and R^{6b} together with the nitrogen atom to which they are attached, form a 4- to 10-membered heterocyclic group optionally substituted by 4- to 10-membered heterocyclic group, a C₃-C₁₅ carbocyclic group optionally substituted by halogen, C₁-C₈-alkyl or hydroxy, or a C₁-C₈-alkyl optionally substituted by 4- to 10-membered heterocyclic group, or a C₃-C₁₅ carbocyclic group optionally substituted by halogen, C₁-C₈-alkyl or hydroxy;
R⁷ is C₃-C₁₅ carbocyclic group or C₁-C₈-alkyl optionally substituted by a C₃-C₁₅ carbocyclic group;

where each C₃-C₁₅-carbocyclic group, unless otherwise specified, can be optionally substituted by at least one halo, cyano, amino, nitro, carboxy, C₁-C₈-alkyl, C₁-C₈-haloalkyl, C₁-C₈-alkoxy, C₁-C₈-cyanoalkyl, C₁-C₈-alkylcarbonyl, C₁-C₈-alkoxycarbonyl, C C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₀-carbocyclic group and a 4- to 10-membered heterocyclic group having at least one ring heteroatom selected from nitrogen, oxygen and sulphur;

and where each 4- to 10-membered heterocyclic group, unless otherwise specified, can be optionally substituted by at least one halo, cyano, oxo, hydroxy, carboxy, nitro, C₁-C₈-alkyl optionally substituted by 4- to 10-membered heterocyclic group, or a C₃-C₁₅ carbocyclic group optionally substituted by halogen, C₁-C₈-alkyl or hydroxy, C₁-C₈-cyanoalkyl, C₁-C₈-alkylcarbonyl, hydroxy-C₁-C₈-alkyl, C₁-C₈-haloalkyl, amino-C₁-C₈-alkyl, amino(hydroxy) C₁-C₈-alkyl and C₁-C₈-alkoxy optionally substituted by aminocarbonyl;

and where each C₆-C₁₅-aromatic carbocyclic group, unless otherwise specified, can be optionally substituted by at least one halo, cyano, amino, nitro, carboxy, C₁-C₈-alkyl, halo-C₁-C₈-alkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₅-carbocyclic group and a 4- to 10-membered heterocyclic group having at least one ring heteroatom selected from nitrogen, oxygen and sulphur; and

m is an integer selected from 1-3.

Compounds of formula (II) may be used to prepare compounds of formula (I) in accordance with known procedures or analogously as hereinafter described in the Examples or Scheme 1.

Compounds of formula (II) may be prepared by reacting a compound of formula (III)

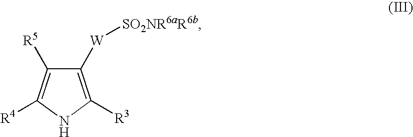

where all symbols are as hereinbefore defined, with a compound of formula (IV)

X-Q-COOR⁷ (IV)

where

X is halogen; and

R⁷ is as hereinbefore defined.

The reaction may be carried out using known procedures for reaction of amines with haloalkylcarboxylic esters, or analogously, as hereinafter described, in the Examples.

The compounds of formula (I) can be prepared, e.g., using the reactions and techniques described below. The reactions may be performed in a solvent appropriate to the reagents and materials employed and suitable for the transformations being effected. It will be understood by those skilled in the art of organic synthesis that the functionality present on the molecule should be consistent with the transformations proposed. T his will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which give examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al., series editors, Pergamon (1995); Comprehensive Organic Synthesis, Trost and Fleming, series editors, Pergamon (1991); and Pavri and Trudell, J Org Chem, Vol. 62, No. 8, pp. 2649-2651 (1997).

The compounds of formula (I) in free form may be converted into salt form, and vice versa, in a conventional manner. The compounds in free or salt form can be obtained in the form of hydrates or solvates containing a solvent used for crystallisation. Compounds of formulae (I) and (II) can be recovered from reaction mixtures and purified in a conventional manner. Isomers, such as enantiomers, may be obtained in a conventional manner, e.g., by fractional crystallisation, chiral HPLC resolution or asymmetric synthesis from correspondingly asymmetrically substituted, e.g., optically active, starting materials.

Generally, compounds described in the scope of this patent application can be synthesized by the route described in Scheme 1.

Scheme 1 depicts the general synthetic scheme when there is a nitrile substituent attached to either the 3- or 4-position of the pyrrole. For instance, in Scheme 1, cinnamonitrile derivative 2 may be prepared by reaction of aldehyde derivative 1 in the presence of an inorganic base, such as sodium hydride, and a phosphonate derivative, preferably diethyl cyanomethylphosphonate in accordance with March, 5^{th} ed., p. 1233. The cinnamonitrile derivative 2 may then be reacted with an (arylsulfonyl)methylisocyanide, such as (p-toluenesulfonyl)methylisocyanide in the presence of a base, as in Pavri and Trudell (1997), supra, to provide pyrrole derivative 3. Pyrrole derivative 3 may be alkylated with an alkyl halide, such as methyl-2-bromoacetate, in the presence of a strong base, such as sodium hydride, to provide compound 4. The nitro functionality of compound 4 may then be reduced in accordance with March, 5^{th} ed, p. 1552 to provide aniline compound 5. The aniline may then be diazotized and converted in situ to the sulfonyl chloride 6, according to March, 5^{th} ed p 937. Compound 6 may then be reacted with an amine to give sulfonamide 7, which is finally hydroysed to afford 8.

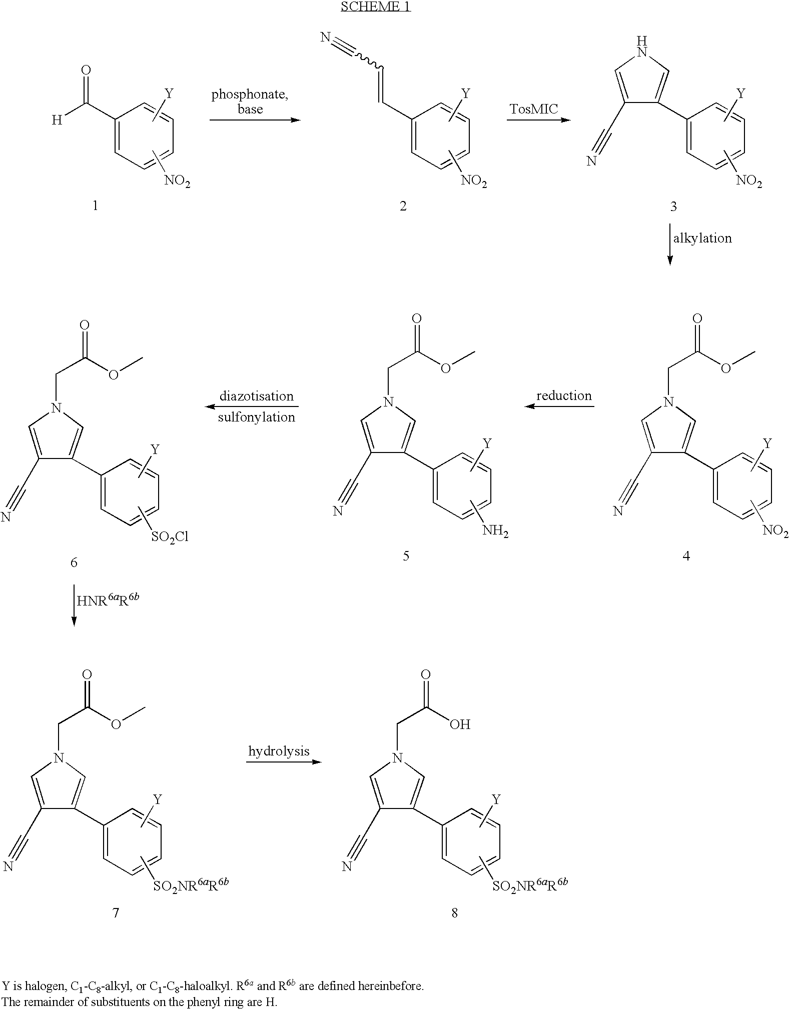

### Pharmaceutical Use and Assay

Compounds of formula (I) and their pharmaceutically acceptable salts, hereinafter referred to alternatively as “agents of the invention”, are useful as pharmaceuticals. In particular, the compounds have good CRTh2 receptor modulator activity and may be tested in the following assays.

### Filtration Binding Assay Protocol

The binding of CRTh2 modulators is determined using membranes prepared from human CRTh2-expressing Chinese Hamster Ovary cells (CHO.K1-CRTh2). To produce cell membranes CHO.K1-CRTh2 cells cultured in roller bottles are harvested using cell dissociation buffer (Invitrogen). The cells are pelleted by centrifugation (167 g, 5 min). The cell pellet is incubated in hypotonic buffer (15 mM Tris-OH, 2 mM MgCl₂, 0.3 mM EDTA, 1 mM EGTA, 1× Complete™ tablet) at 4° C. for 30 minutes. At 4° C. cells are homogenized using a Polytron®) (IKA Ultra Turrax T25) for 5 bursts of 1 second. The homogenate is centrifuged (Beckman Optima TM TL Ultracentrifuge, 48000 g, 30 minutes at 4° C.). The supernatant is discarded and the membrane pellet re-suspended in homogenisation buffer (75 mM Tris-OH, 12.5 mM MgCl₂, 0.3 mM EDTA, 1 mM EGTA, 250 mM Sucrose, 1× Complete™ tablet. Membrane preparations are aliquoted and stored at 80° C. The protein content is estimated using Bradford Protein Assay Dye (Bio Rad).

The binding of [³H]-PGD₂ (157 Ci/mmol) to CHO.K1-CRTh2 membranes is determined in the absence (total binding) and presence (non-specific binding) of unlabelled PGD₂ (1 μM). Subtraction of the cpm (counts per minute) of [³H]-PGD₂ binding in presence of excess unlabelled PGD₂ from that observed in the absence of excess unlabelled PGD₂ is defined as specific binding. Active CRTh2 antagonists are able to compete with [³H]-PGD₂ for binding to the CRTh2 receptor and are identified in a decrease in the number of cpm bound.

The assay is performed in Greiner U-bottomed 96 well-plates, in a final volume of 100 μL per well. CHO.K1-CRTh2 membranes were diluted in assay buffer (10 mM HEPES-KOH (pH 7.4), 1 mM EDTA and 10 mM MnCl₂ and 10 μg are added to each well [³H]-PGD₂ is diluted in assay buffer and added to each well at a final concentration of 2.5 nM. To determine non-specific binding, [³H]-PGD₂ binding to the CRTh2 receptor is competed with using unlabelled PGD₂ at a final well concentration of 1 μM. The experiment is done in triplicate, with reagents added to the wells as follows:

25 μL assay buffer for total binding or

25 μL PGD₂ to determine non-specific binding

25 μL [³H]PGD₂

50 μL membranes

25 μL test compound in DMSO/assay buffer

The plates are incubated at room temperature on a shaker for 1 hour, and then harvested (Tomtec Harvester 9600) onto GF/C filter plates using wash buffer (10 mM HEPES-KOH, pH 7.4). The plate is dried for 2 hours, prior to addition of Micro-Scint 20™ (50 μL) and sealing with TopSeal-S™. Plates are then counted using a Packard Top Count instrument, Plates are then read on the Packard Topcount with the 3H Scintillation program (1 min./well).

Ki (dissociation constant for the inhibition) values for the CRTh2 antagonists are reported. Ki values are determined using Sigma Plots software, using the Cheng-Prussoff equation.

Ki=IC₅₀/1+[S]/Kd

where S is the concentration of radioligand and Kd is the dissociation constant.
CRTH2 cAMP Functional Assay Protocol

This assay is conducted in CHO.K1-CRTh2 cells. cAMP is generated in the cell by stimulating cells with 5 μM forskolin, an adenylate cyclase activator. PGD₂ is added to activate the CRTh2 receptor which results in the attenuation of the forskolin-induced cAMP accumulation. Potential CRTh2 antagonists are tested for their ability to inhibit the PGD₂-mediated attenuation of the forskolin-induced cAMP accumulation in CHO.K1-CRTh2 cells.

For each concentration value on the dose-response curve, test compounds are prepared in assay stimulation buffer (HBSS, 5 mM HEPES, 10 μM IBMX±0.1% human serum albumin) containing DMSO (3% vol/vol) and 5 μL/well is added to an assay plate (384 well white optiplate).

CHO.K1-CRTh2 cultured in tissue culture flasks are washed with PBS and harvested with dissociation buffer. Cells are washed with PBS and re-suspended in stimulation buffer to a concentration of 0.4×10⁶/mL and added to the assay plate (10 μL/well).

The assay plate is incubated at room temperature on a shaker for 15 minutes.

A mix of agonist (10 nM Prostaglandin D and 5 μM forskolin is prepared in assay stimulation buffer and added to the assay plate (5 μL/well).

In addition, a cAMP standard is serially diluted in assay stimulation buffer and added to separate empty wells on the assay plate (20 μL/well). The cAMP standard allows for the quantification of cAMP generated in CHO.K1-CRTH2 cells.

The assay plate is incubated at room temperature on a shaker for 60 minutes.

Cell lysis buffer (Lysis buffer Milli-Q H₂0, 5 mM HEPES, 0.3% Tween-20, 0.1% human serum albumin) is added to a bead mix (containing Alphascreen™ anti-cAMP acceptor beads 0.06 units/μL, Alphascreen™streptavidin-coated donor beads 0.06 units/mL, biotinylated cAMP 0.06 units/μL, 10 μM IBMX) is prepared under darkened conditions 60 minutes prior to addition to the assay plate. The resulting lysis mix is added to all wells of the assay plate (40 μL/well).

The assay plate is sealed with Topseal-S™ and incubated in the dark at room temperature on a shaker for 45 minutes. The plate is then counted using a Packard Fusion™ instrument.

The resulting counts per minute are converted to nM cAMP by using the prepared cAMP standard curve. IC₅₀ values (concentration of CRTh2 antagonist required to inhibit 50% of the PGD₂-mediated attenuation of forskolin induced cAMP accumulation in CHO.K1-CRTh2 cells) are then determined using Prism™ software.

Compounds of the Examples, herein below, generally have Ki values in the filtration binding assay below 10 μM. For example, the compounds of Examples 3, 8 and 9 have Ki values of 0.017, 0.002 and 0.049 μM, respectively.

Compounds of the Examples, herein below, generally have IC₅₀ values in the functional assay below 10 μM. For example, the compounds of Examples 3, 8 and 9 have IC₅₀ values of 0.002, 0.005 and 0.026 μM, respectively.

Compounds of formula (I), in free or salt form, are modulators of the G-protein-coupled receptor CRTh2, expressed on Th2 cells, eosinophils and basophils. PGD₂ is the natural ligand for CRTh2. Thus, antagonists which inhibit the binding of CRTh2 and PGD₂ are useful in the treatment of allergic and anti-inflammatory conditions. Treatment in accordance with the invention may be symptomatic or prophylactic.

Accordingly, agents of the invention are useful in the treatment of inflammatory or obstructive airways diseases, resulting, e.g., in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitis asthma, exercise induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., corticosteroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 AM, i.e., at a time normally substantially distant from any previously administered symptomatic asthma therapy.

Other inflammatory or obstructive airways diseases and conditions to which the present invention is applicable include acute lung injury (ALI), adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis including, e.g., aluminosis, anthracosis, asbestosis, chalicosis, ptilosis, siderosis, silicosis, tabacosis and byssinosis.

Having regard to their antiinflammatory activity, in particular, in relation to inhibition of eosinophil activation, agents of the invention are also useful in the treatment of eosinophil related disorders, e.g., eosinophilia, in particular, eosinophils-related disorders of the airways, e.g., involving morbid eosinophilic infiltration of pulmonary tissues including hypereosinophilia as it effects the airways and/or lungs, as well as, e.g., eosinophil-related disorders of the airways consequential or concomitant to Löffer's syndrome; eosinophilic pneumonia; parasitic, in particular, metazoan, infestation including tropical eosinophilia; bronchopulmonary aspergillosis; polyarteritis nodosa including Churg-Strauss syndrome; eosinophilic granuloma; and eosinophil-related disorders affecting the airways occasioned by drug-reaction.

Agents of the invention are also useful in the treatment of inflammatory or allergic conditions of the skin, e.g., psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita and other inflammatory or allergic conditions of the skin.

Agents of the invention may also be used for the treatment of other diseases or conditions, in particular, diseases or conditions having an inflammatory component, e.g., treatment of diseases and conditions of the eye, such as conjunctivitis, keratoconjunctivitis sicca and vernal conjunctivitis; diseases affecting the nose including allergic rhinitis; and inflammatory disease, in which autoimmune reactions are implicated or having an autoimmune component or aetiology, including autoimmune hematological disorders, e.g., hemolytic anemia, aplastic anaemia, pure red cell anaemia and idiopathic thrombocytopenia; systemic lupus erythematosus; polychondritis; sclerodoma; Wegener granulamatosis; dermatomyositis; chronic active hepatitis; myasthenia gravis; Steven-Johnson syndrome; idiopathic sprue; autoimmune inflammatory bowel disease, e.g., ulcerative colitis and Crohn's disease; endocrine opthalmopathy; Grave's disease; sarcoidosis; alveolitis; chronic hypersensitivity pneumonitis; multiple sclerosis; primary billiary cirrhosis; uveitis (anterior and posterior); keratoconjunctivitis sicca and vernal keratoconjunctivitis; interstitial lung fibrosis; psoriatic arthritis; and glomerulonephritis, with and without nephrotic syndrome, e.g., including idiopathic nephrotic syndrome or minal change nephropathy.

Other diseases or conditions which may be treated with agents of the invention include septic shock; rheumatoid arthritis; osteoarthritis; proliferative diseases, such as cancer; atherosclerosis; allograft rejection following transplantation; stroke; obesity; restenosis; diabetes, e.g., diabetes mellitus type I (juvenile diabetes) and diabetes mellitus type II; diarrheal diseases; ischemia/reperfusion injuries; retinopathy, such as diabetic retinopathy or hyperbaric oxygen induced retinopathy; and conditions characterized by elevated intraocular pressure or secretion of ocular aqueous humor, such as glaucoma.

Other diseases or conditions which may be treated with agents of the invention include neuropathic pain as described in WO 05/102338.

The effectiveness of an agent of the invention in inhibiting inflammatory conditions, e.g., in inflammatory airways diseases, may be demonstrated in an animal model, e.g., a mouse or rat model, of airways inflammation or other inflammatory conditions, e.g., as described by Szarka et al., J Immunol Methods, Vol. 202, pp. 49-57 (1997); Renzi et al., Am Rev Respir Dis, Vol. 148, pp. 932-939 (1993); Tsuyuki et al., J Clin Invest, Vol. 96, pp. 2924-2931 (1995); Cernadas et al., Am J Respir Cell Mol Biol, Vol. 20, pp. 1-8 (1999); and Williams and Galli, J Exp Med, Vol. 192, pp. 455-462 (2000).

The agents of the invention are also useful as co-therapeutic agents for use in combination with other drug substances, such as antiinflammatory, bronchodilatory or antihistamine drug substances, particularly in the treatment of obstructive or inflammatory airways diseases, such as those mentioned hereinbefore, e.g., as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance. Accordingly the invention includes a combination of an agent of the invention as hereinbefore described with an anti-inflammatory, bronchodilatory, antihistamine or anti-tussive drug substance, said agent of the invention and said drug substance being in the same or different pharmaceutical composition.

Such anti-inflammatory drugs include steroids, in particular, glucocorticosteroids, such as budesonide, beclamethasone dipropionate, fluticasone propionate, ciclesonide or mometasone furoate; or steroids, described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445 and WO 03/072592, WO 04/039827, WO 04/066920; non-steroidal glucocorticoid receptor agonists, such as those described in WO 00/00531, WO 02/10143, DE 10261874, WO 03/082280, WO 03/082787, WO 03/104195, WO 03/101932, WO 04/019935, WO 04/018429, WO 04/063163, WO 04/005229, WO 03/086294 and WO 04/26248, WO 04/071389; LTB4 antagonists, such as those described in U.S. Pat. No. 5,451,700; LTD4 antagonists, such as montelukast and zafirlukast; PDE4 inhibitors, such as cilomilast (Ariflo®GlaxoSmithKline), Roflumilast (Byk Gulden), V-11294A (Napp), BAY19-8004 (Bayer), SCH-351591 (Schering-Plough), Arofylline (Almirall Prodesfarma), PD189659 (Parke-Davis), AWD-12-281 (Asta Medica), CDC-801 (Celgene), SelCID(TM) CC-10004 (Celgene), KW-4490 (Kyowa Hakko Kogyo), WO 03/104204, WO 03/104205, WO 04/000814, WO 04/000839 and WO 04/005258 (Merck), as well as those described in WO 98/18796 and WO 03/39544; A2a agonists, such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462 and WO 03/086408; A2b antagonists, such as those described in WO 02/42298; and beta (β)-2-adrenoceptor agonists, such as albuterol (salbutamol), metaproterenol, terbutaline, salmeterol, fenoterol, procaterol, and especially, formoterol and pharmaceutically acceptable salts thereof, and compounds (in free or salt or solvate form) of formula (I) of WO 00/75114, which document is incorporated herein by reference, preferably compounds of the Examples thereof, especially a compound of formula

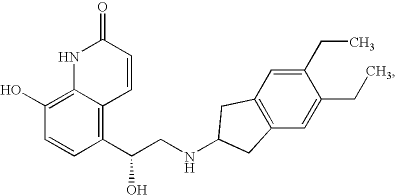

and pharmaceutically acceptable salts thereof, as well as compounds (in free or salt or solvate form) of formula (I) of WO 04/16601. Further β-2-adrenoreceptor agonists include compounds of JP 05025045, WO 93/18007, WO 99/64035, U.S. Patent No. 2002/0055651, WO 01/42193, WO 01/83462, WO 02/66422, WO 02/70490, WO 02/76933, WO 03/024439, WO 03/072539, WO 03/042160, WO 03/091204, WO 03/042164, WO 03/099764, WO 04/016578, WO 04/022547, WO 04/032921, WO 04/037773, WO 04/037807, WO 04/039762, WO 04/039766, WO 04/045618, WO 04/046083, WO 04/033412, WO 04/037768, WO 04/037773 and EP 1440966.

Such bronchodilatory drugs include anticholinergic or antimuscarinic agents, in particular, ipratropium bromide, oxitropium bromide, tiotropium salts and CHF 4226 (Chiesi), but also those described in WO 04/096800, WO 01/04118, WO 02/51841, WO 02/53564, WO 03/00840, WO 03/87094, WO 04/05285, WO 02/00652, WO 03/53966, EP 0424021, U.S. Pat. No. 5,171,744, U.S. Pat. No. 3,714,357 and WO 03/33495.

Such co-therapeutic antihistamine drug substances include cetirizine hydrochloride, acetaminophen, clemastine fumarate, promethazine, loratidine, desloratidine, diphenhydramine and fexofenadine hydrochloride.

Combinations of agents of the invention and steroids, β-2 agonists, PDE4 inhibitors or LTD4 antagonists may be used, e.g., in the treatment of COPD or, particularly, asthma. Combinations of agents of the invention and anticholinergic or antimuscarinic agents, PDE4 inhibitors, dopamine receptor agonists or LTB4 antagonists may be used, e.g., in the treatment of asthma or, particularly, COPD.

Other useful combinations of agents of the invention with anti-inflammatory drugs are those with antagonists of chemokine receptors, e.g., CCR-1, CCR-2, CCR-3, CCR-4, CCR-5, CCR-6, CCR-7, CCR-8, CCR-9, CCR-10, CXCR1, CXCR2, CXCR3, CXCR4 and CXCR5; particularly useful are CCR-3 antagonists, such as those described in WO 02/026723, especially 4-{3-[(S)-4-(3,4-dichlorobenzyl)-morpholin-2-ylmethy]-ureidomethyl}-benzamide and those described in WO 03/077907, WO 03/007939 and WO 02/102775.

Also especially useful are CCR-5 antagonists, such as Schering-Plough antagonists SC-351125, SCH-55700 and SCH-D; Takeda antagonists, such as N-[[4-[[[6,7-dihydro-2-(4-methylphenyl)-5H-benzo-cyclohepten-8-yl]carbonyl]amino]phenyl]methyl]tetrahydro-N,N-dimethyl-2H-pyran-4-aminium chloride (TAK-770); and CCR-5 antagonists, described in U.S. Pat. No. 6,166,037, WO 00/66558 and WO 00/66559.

The agents of the invention may be administered by any appropriate route, e.g., orally, e.g., in the form of a tablet or capsule; parenterally, e.g., intravenously; by inhalation, e.g., in the treatment of inflammatory or obstructive airways disease; intranasally, e.g., in the treatment of allergic rhinitis; to pically to the skin, e.g., in the treatment of atopic dermatitis; or rectally, e.g., in the treatment of inflammatory bowel disease.

The present invention also provides a pharmaceutical composition comprising a compound of formula (I), in free form or in the form of a pharmaceutically acceptable salt, optionally together with a pharmaceutically acceptable diluent or carrier therefore. The composition may contain a co-therapeutic agent, such as an anti-inflammatory, bronchodilatory or antihistamine drug, as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

The present invention also provides for the use of a compound of the present invention in any of the aforementioned embodiments, in free or pharmaceutically acceptable salt form, for the manufacture of a medicament for the treatment of an inflammatory or allergic condition, particularly an inflammatory or obstructive airways disease.

The present invention also provides a method for treating or preventing inflammatory or allergic conditions comprising administering to a patient in need thereof a therapeutically effective amount of a compound of the present invention, in free or a pharmaceutically acceptable salt form.

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight); and/or one or more surfactants, such as oleic acid or sorbitan trioleate; and/or one or more bulking agents, such as lactose. When the composition comprises a dry powder formulation, it preferably contains, e.g., the compound of formula (I) having a particle diameter up to 10 microns, optionally together with a diluent or carrier, such as lactose, of the desired particle size distribution and a compound that helps to protect against product performance deterioration due to moisture. When the composition comprises a nebulized formulation, it preferably contains, e.g., the compound of formula (I), either dissolved or suspended, in a vehicle containing water, a co-solvent, such as ethanol or propylene glycol and a stabilizer, which may be a surfactant.

The invention includes:
(a) an agent of the invention in inhalable form, e.g., in an aerosol or other atomizable composition or in inhalable particulate, e.g., micronized form;
(b) an inhalable medicament comprising an agent of the invention in inhalable form;
(c) a pharmaceutical product comprising such an agent of the invention in inhalable form in association with an inhalation device; and
(d) an inhalation device containing an agent of the invention in inhalable form.

Dosages of agents of the invention employed in practicing the present invention will of course vary depending, e.g., on the particular condition to be treated, the effect desired and the mode of administration. In general, suitable daily dosages for oral administration are of the order of 0.01-100 mg/kg.

The invention is illustrated by the following Examples.

### EXAMPLES

### General Conditions

LCMS are recorded on an Agilent 1100 LC system with a Waters Xterra MS C18 4.6×100 5 μM column, eluting with 5-95% 10 mM aqueous ammonium bicarbonate in acetonitrile over 2.5 minutes, with negative ion electrospray ionization or 5-95% water+0.1% TFA in acetonitrile with positive ion electrospray ionization. [M+H]⁺ and [M−H]⁻ refer to monoisotopic molecular weights.

### Abbreviations

| | | | |
|---|---|---|---|
| AcOH | acetic acid | MeOH | methanol |
| CuCl₂ | copper (II) chloride | MgSO₄ | magnesium sulfate |
| DCM | dichloromethane | NaH | sodium hydride |
| DIBAL | diisobutylaluminium | NaOH | sodium hydroxide |
| | hydride | | |
| DMF | dimethylformamide | Na₂SO₄ | sodium sulfate |
| DMSO | dimethyl sulfoxide | PS-CDI | polymer supported |
| Et₃N | triethylamine | | carbodiimide |
| EtOAc | ethyl acetate | SO₂ | sulphur dioxide |
| EtOH | ethanol | RT | room temperature |
| Fe | iron | t-BuOK | potassium tert-butoxide |
| HCl | hydrochloric acid | THF | tetrahydrofuran |
| HOBt | 1- | TosMIC | (p-toluenesulfonyl) |
| | hydroxybenzotriazole | | |
| H₂O | water | | methylisocyanide |
| HPLC | high performance | SnCl₂,2H₂O | Tin(II) chloride dihydrate |
| | liquid chromatography | | |
| | | PS-DIEA | Diisopropylaminomethyl- |
| LiOH | lithium hydroxide | | polystyrene |
| MeCN | acetonitrile | | |

The following examples have been prepared using the process described herein.

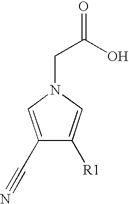

| | | [M + |
|---|---|---|
| | | H]+/ |
| Exam- | | [M − |
| ple | R₁ | H]⁻ |
| 1 | 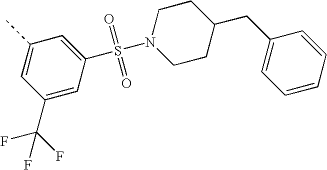 | 532 |
| 2 | 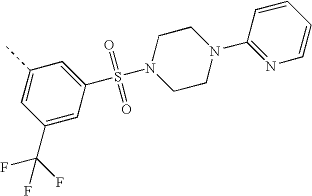 | 520 |
| 3 | 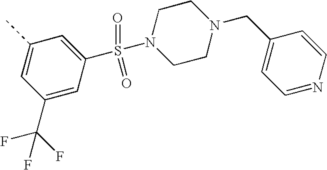 | 534 |
| 4 | 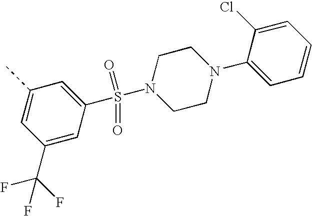 | 553 |
| 5 | 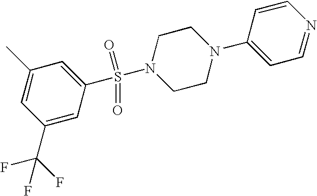 | 520 |
| 6 | 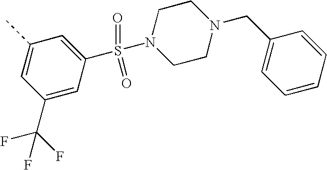 | 533 |
| 7 | 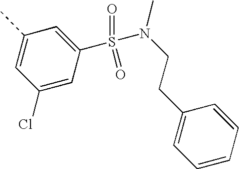 | 458 |
| 8 | 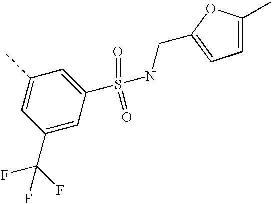 | 485 |
| 9 | 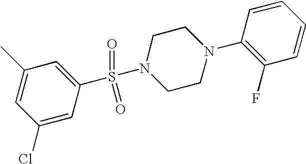 | 502/504 |
| 10 | 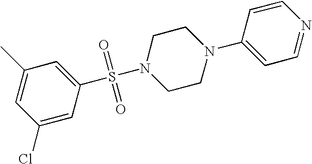 | 486/488 |
| 11 | 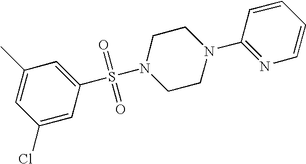 | 486 |
| 12 | 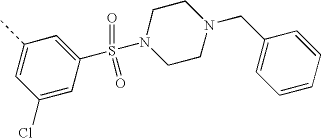 | 499 |
| 13 | 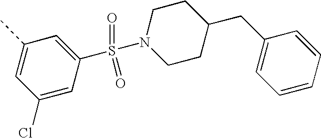 | 497/499 |
| 14 | 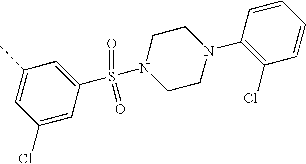 | 518/520 |
| 15 | 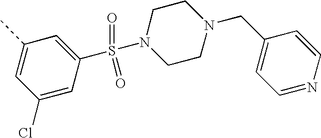 | 500/502 |

### Example 1

### Preparation of {3-[3-(4-Benzyl-piperidine-1-sulfonyl)-5-trifluoromethyl-phenyl]-4-cyano-pyrrol-1-yl}-acetic acid

### a) (3-Nitro-5-trifluoromethyl-phenyl)-methanol

To a solution of commercially available 3-nitro-5-(trifluoromethyl)benzoic acid (85 g, 0.362 mol) in dry THF (340 ml) at 0° C. is added 1M BH₃ in THF (542 ml) over 30 minutes. The resulting reaction mixture is stirred at 0° C. for 40 minutes then allowed to warm to room temperature and stirred overnight. The reaction mixture is cooled to 0° C. and carefully quenched with water (220 ml) maintaining the reaction temperature below 10° C. The reaction mixture is allowed to warm to room temperature, the solvent is removed under reduced pressure and the resulting crude residue is partitioned between EtOAc and 1M NaOH solution. The organic layer is separated, the aqueous layer extracted with EtOAc and the combined organic layers are washed with water, brine, dried over MgSO₄. After filtration the solvent is evaporated under reduced pressure and dried under high vacuum to give (3-nitro-5-trifluoromethyl-phenyl)-methanol as a yellow/orange oil.

### b) 3-Nitro-5-trifluoromethyl-benzaldehyde

To a solution of oxalyl chloride (44.2 ml, 0.522 mol) in DCM (400 ml) at −75° C. is added a solution of dry DMSO (82.4 ml, 1.16 mol) in DCM (400 ml) dropwise maintaining the reaction temperature below −70° C. The reaction mixture is stirred at −78° C. for 60 minutes. A solution of (3-nitro-5-trifluoromethyl-phenyl)-methanol (51.3 g, 0.232 mol) in DCM (400 ml) is added dropwise maintaining the reaction temperature below −70° C., over 20 minutes. The reaction mixture is stirred at −78° C. for 80 minutes. Triethylamine (166 ml, 1.18 mol) is added dropwise over 20 minutes, maintaining the reaction temperature below −70° C. The reaction mixture is allowed to warm to room temperature slowly and stirred overnight. Water is added to the reaction mixture, the aqueous layer separated and extracted with DCM. The combined organic layers are washed with water, brine, dried over MgSO₄ and decolorized with charcoal for 30 minutes. The organic layer is filtered, the solvent is evaporated under reduced pressure, dried under high vacuum to give the crude 3-nitro-5-trifluoromethyl-benzaldehyde as orange crystals; [M−H]⁻ 218.

### c) 3-(3-Nitro-5-trifluoromethyl-phenyl)-acrylonitrile

To a suspension of sodium hydride (60% dispersion in oil, 10.0 g, 0.250 mol) in dry THF (460 ml) is added at 5° C. a solution of diethyl cyanomethylphosphonate (39.4 ml, 0.250 mol) in THF (180 ml) dropwise over 20 minutes, maintaining the reaction temperature below 10° C. The suspension is stirred at 5° C. for 60 minutes. A solution of 3-nitro-5-trifluoromethyl-benzaldehyde (45.7 g, 0.209 mol) in THF (320 ml) is added dropwise over 20 minutes, maintaining the reaction temperature below 10° C. The reaction mixture is allowed to warm to room temperature and stirred overnight. Water is added, the solvent is evaporated off, the residue is partitioned between EtOAc and water. The aqueous layer is separated and extracted with EtOAc, the combined organic layers are washed with water, brine, dried over MgSO₄ and decolorized with charcoal. The organic layer is filtered, the solvent is evaporated under reduced pressure to give 3-(3-nitro-5-trifluoromethyl-phenyl)-acrylonitrile; [M−H]⁻ 241.

### d) 4-(3-Nitro-5-trifluoromethyl-phenyl)-1H-pyrrole-3-carbonitrile

To a suspension of sodium hydride (60% dispersion in oil, 9.79 g, 0.245 mol) in dry THF (1500 ml) is added at 0° C. a solution of 3-(3-nitro-5-trifluoromethyl-phenyl)-acrylonitrile (49.4 g, 0.204 mol) and TosMIC (47.8 g, 0.245 mol) in THF (750 ml) dropwise over 40 minutes, maintaining the reaction temperature below 5° C. The reaction mixture is allowed to warm to room temperature and stirred overnight. Water (120 ml) is added, the solvent evaporated and the residue is partitioned between DCM and water. The aqueous layer is separated, extracted with DCM, the combined organic layers are washed with water, brine, dried over MgSO₄ and decolorized with charcoal. The organic layer is filtered, the solvent is evaporated and dried under high vacuum overnight to give the crude product as a very dark brown oily solid. The oily solid is triturated in DCM (40 ml) for 30 minutes, insoluble solid filtered off, washed with DCM and dried in vacuum at 40° C. to give 4-(3-nitro-5-trifluoromethyl-phenyl)-1H-pyrrole-3-carbonitrile; [M−H]⁻ 280.

### e) [3-Cyano-4-(3-nitro-5-trifluoromethyl-phenyl)-pyrrol-1-yl]-acetic acid methyl ester

To a suspension of sodium hydride (60% dispersion in oil, 2.75 g, 0.069 mol) in dry DMF (150 ml) is added at 0° C. a solution of 4-(3-nitro-5-trifluoromethyl-phenyl)-1H-pyrrole-3-carbonitrile (12.92 g, 0.046 mol) in DMF (100 ml) dropwise over 35 minutes, maintaining the reaction temperature below 5° C. The reaction mixture is allowed to warm to room temperature, stirred for 60 minutes and then cooled to 5° C. Methyl bromoacetate (3.57 ml, 0.046 mol) is added dropwise maintaining the reaction temperature below 10° C. The reaction mixture is allowed to warm to room temperature and stirred for 3 hours. Further methyl bromoacetate (0.72 ml, 0.0098 mol) is added and stirred for 50 minutes. Water is added over 15 minutes, the solid is filtered off, washed with water and dried overnight in vacuum at 40° C. over P₂O₅ to give [3-cyano-4-(3-nitro-5-trifluoromethyl-phenyl)-pyrrol-1-yl]acetic acid methyl ester; [M−H]⁻ 352.

### f) 3-(3-Amino-5-trifluoromethyl-phenyl)-4-cyano-pyrrol-1-yl]-acetic acid methyl ester

[3-Cyano-4-(3-nitro-5-trifluoromethyl-phenyl)-pyrrol-1-yl]-acetic acid methyl ester (1.0 g, 2.83 mmol) in MeOH (4 ml) and AcOH (7 ml) is treated with Fe (325 mesh, 0.791 g, 14.16 mmol) and the reaction mixture is heated at 80° C. for 30 minutes giving a brown solution. The reaction mixture is allowed to cool to room temperature and poured into water. The pH of the solution is adjusted to pH 7-8 by addition of saturated sodium hydrogen carbonate solution, the resulting emulsion is filtered and extracted with EtOAc. The combined organic layers are washed with brine, dried over MgSO₄, the solvent is removed under reduced pressure to give an orange/brown oil. The crude product is purified by flash chromatography (gradient from isohexane to 4:6 isohexane:EtOAc) to afford the titled compound as an orange oily solid; [M−H]⁻ 322.

### g) [3-(3-Chlorosulfonyl-5-trifluoromethyl-phenyl)-4-cyano-pyrrol-1-yl]acetic acid-methyl ester

To a solution of 3-(3-amino-5-trifluoromethyl-phenyl)-4-cyano-pyrrol-1-yl]-acetic acid methyl ester (0.200 g, 0.6 mmol) is added at 0° C. AcOH (2 ml) and conc HCl (1 ml). Then the solution is treated dropwise with a solution of sodium nitrite (42.7 mg, 0.62 mmol) in water (0.5 ml). After stirring at 0° C. for 1.5 hours, the bright yellow reaction mixture is added portionwise to a stirred solution of SO₂/AcOH/CuCl₂(H₂O (20 ml) (the preparation of the reagent is described below). The reaction mixture is allowed to warm to room temperature, is stirred for 5 hours, then poured into water (200 ml) and extracted with EtOAc. The combined organic layers are washed with water followed by brine and dried over MgSO₄. After filtration the solvent is removed under reduced pressure to give a pink solid. The crude product is purified by flash chromatography (gradient from isohexane to 3:7 isohexane:EtOAc), to afford the titled compound as a pink solid; [M+H₂O]⁺424.

Preparation of the reagent SO₂/AcOH/CuCl₂/H₂O:

According to the reported procedure (E. E. Gilbert, Synthesis, 1-10, p 6 (1969), glacial AcOH (100 mL) vigorously stirred at RT is treated by bubbling SO₂ gas. Once a saturated solution is achieved (approximately 10 g per 100 mL), the solution is treated with CuCl₂ (4 g) in water (5 mL). The resulting mixture is allowed to settle to give a green solution.

### h) {3-[3-(4-Benzyl-piperidine-1-sulfonyl)-5-trifluoromethyl-phenyl]-4-cyano-pyrrol-1-yl}acetic acid methyl ester

To PS-DIEA (90.2 mg, 0.33 mmol) in THF (1 ml) is added at room temperature a solution of [3-(3-chlorosulfonyl-5-trifluoromethyl-phenyl)-4-cyano-pyrrol-1-yl]-acetic acid methyl ester (45.0 mg, 0.11 mmol) in THF (1 ml), followed by the addition of 4-benzyl-piperidine (19.7 μl, 0.11 mmol). The reaction mixture is stirred at room temperature for 1.5 hours. The reaction mixture is filtered and the solid washed with THF, EtOAc and MeOH. The filtrate is evaporated under reduced pressure to give a pale pink solid. The crude product is purified by flash chromatography (gradient from isohexane to 0:1 isohexane:EtOAc), to afford the titled compound as a white solid; [M+H]⁺ 546.

### i) {3-[3-(4-Benzyl-piperidine-1-sulfonyl)-5-trifluoromethyl-phenyl]-4-cyano-pyrrol-1-yl}-acetic acid

A solution of {3-[3-(4-benzyl-piperidine-1-sulfonyl)-5-trifluoromethyl-phenyl]-4-cyano-pyrrol-1-yl}acetic acid methyl ester (51.1 mg, 0.09 mmol) in THF (1 ml) and water (1 ml) is treated at room temperature with NaOH solution (1M, 94 μl, 0.09 mmol) and the resulting pale yellow reaction mixture is stirred for 4 hours. The solvent is evaporated under reduced pressure to give a residue. The residue is treated with H₂O, acidified to pH 1 using 1M HCl solution, extracted with DCM, the solvent is evaporated under reduced pressure and dried under vacuum to give the titled compound as a pale yellow solid; [M+H]⁺532.

### Examples 2 to 6

These examples namely,
{3 Cyano 4-[3-(4-pyridin-2-yl-piperazine-1-sulfonyl)-5-trifluoromethyl-phenyl]-pyrrol-1-yl}-acetic acid, sodium salt (Example 2),
{3-Cyano-4-[3-(4-pyridin-4-ylmethyl-piperazine-1-sulfonyl)-5-trifluoromethyl-phenyl]-pyrrol-1-yl}-acetic acid, sodium salt (Example 3),
(3-{3-[4-(2-Chloro-phenyl)-piperazine-1-sulfonyl]-5-trifluoromethyl-phenyl}-4-cyano-pyrrol-1-yl)-acetic acid, sodium salt (Example 4),
{3-Cyano-4-[3-(4-pyridin-4-yl-piperazine-1-sulfonyl)-5-trifluoromethyl-phenyl]-pyrrol-1-yl}-acetic acid, sodium salt (Example 5) and
{3-[3-(4-Benzyl-piperazine-1-sulfonyl)-5-trifluoromethyl-phenyl]-4-cyano-pyrrol-1-yl}-acetic acid, sodium salt (Example 6)
   are prepared by similar processes as that described in Example 1

### Example 7

### Preparation of {3-[3-Chloro-5-(methyl-phenethyl-sulfamoyl)-phenyl]-4-cyano-pyrrol-1-yl}-acetic acid

### a) [3-(3-Amino-5-chloro-phenyl)-4-cyano-pyrrol-1-yl]-acetic acid methyl ester

[3-(3-Amino-5-chloro-phenyl)-4-cyano-pyrrol-1-yl]-acetic acid methyl ester is prepared analogously to 3-(3-amino-5-trifluoromethyl-phenyl)-4-cyano-pyrrol-1-yl]-acetic acid methyl ester (an intermediate in Example 1) by replacing [3-cyano-4-(3-nitro-5-trifluoromethyl-phenyl)-pyrrol-1-yl]-acetic acid methyl ester with [3-(3-chloro-5-nitro-phenyl)-4-cyano-pyrrol-1-yl]acetic acid methyl ester; [M−H]⁻ 288.

### b) [3-(3-Chloro-5-chlorosulfonyl-phenyl)-4-cyano-pyrrol-1-yl]-acetic acid methyl ester

A solution of [3-(3-amino-5-chloro-phenyl)-4-cyano-pyrrol-1-yl]-acetic acid methyl ester (0.615 g, 2.12 mmol) in AcOH (10 ml) and conc HCl (2 ml) is treated at 0° C. dropwise with a solution of sodium nitrite (0.1464 g, 2.12 mmol) in water (1 ml). After stirring at 0° C. for 50 minutes, the reaction mixture is added dropwise to a stirred solution of SO₂/AcOH/CuCl₂/H₂O (30 ml) (the preparation of the reagent is described herein) over 30 minutes. The reaction mixture is allowed to warm to room temperature and is stirred overnight. The reaction mixture is then poured into water (150 ml) and extracted with EtOAc. The combined organic layers are washed with water followed by brine and dried over MgSO₄. After filtration the solvent is removed under reduced pressure to give a red oily solid as a mixture of the titled compound and [3-(3-chloro-5-chlorosulfonyl-phenyl)-4-cyano-pyrrol-1-yl]-acetic acid. The mixture is used without further purification in the next step.

### c) {3-[3-Chloro-5-(methyl-phenethyl-sulfamoyl)-phenyl]-cyano-pyrrol-1-yl}acetic acid

To a solution of a mixture of [3-(3-chloro-5-chlorosulfonyl-phenyl)-4-cyano-pyrrol-1-yl]-acetic acid methyl ester and [3-(3-chloro-5-chlorosulfonyl-phenyl)-4-cyano-pyrrol-1-yl]-acetic acid (0.149 g, ˜0.4 mmol) in dry THF (14 ml) is added Et₃N (67 μl 0.48 mmol) followed by N-methyl-2-phenylmethylamine (65 mg, 0.48 mmol). The reaction mixture is stirred at room temperature over the weekend. The reaction mixture is treated with LiOH solution (1M, 0.8 ml, 0.8 mmol) at room temperature and the resulting reaction mixture is stirred for 1 hour. The reaction mixture is washed with DCM, the aqueous phase acidified to pH 4-5 using 1M HCl solution. The aqueous phase is extracted with DCM and combined organics dried over MgSO₄. After filtration the solvent is removed under reduced pressure to give a crude residue which is triturated with EtOAc and isohexane. The solid is filtered, washed with isohexane, dried under vacuum to give the titled compound as a cream solid; [M+H]⁺458.

### Examples 8

### (3-Cyano-4-{3-[(5-methyl-furan-2-ylmethyl)-sulfamoyl]-5-trifluoromethyl-phenyl}-pyrrol-1-yl)-acetic acid

The titled compound is prepared analogously to {3-[3-Chloro-5-(methyl-phenethyl-sulfamoyl)-phenyl]-4-cyano-pyrrol-1-yl}-acetic acid by replacing a mixture of [3-(3-chloro-5-chlorosulfonyl-phenyl)-4-cyano-pyrrol-1-yl]-acetic acid methyl ester and [3-(3-chloro-5-chlorosulfonyl-phenyl)-4-cyano-pyrrol-1-yl]-acetic acid (Intermediate 7c) with a mixture of [3-(3-chlorosulfonyl-5-trifluoromethyl-phenyl)-4-cyano-pyrrol-1-yl]-acetic acid-methyl ester and [3-(3-chlorosulfonyl-5-trifluoromethyl-phenyl)-4-cyano-pyrrol-1-yl]-acetic acid.

### Example 9

### Preparation of (3-{3-Chloro-5-[4-(2-fluoro-phenyl)-piperazine-1-sulfonyl]-phenyl}-4-cyano-pyrrol-1-yl)-acetic acid, sodium salt

### a) 3-Chloro-5-nitro-benzoic acid methyl ester

To a solution of commercially available 3-amino-5-nitrobenzoic acid methyl ester (32.0 g, 0.163 mol) in conc HCl (332 ml) and AcOH (464 ml) at 0° C. is added NaNO₂ (11.28 g, 0.163 mol) in water (20 ml) dropwise over 20 minutes, maintaining, the reaction temperature below 0° C. The reaction mixture is stirred at 0° C. for 1 hour. The reaction mixture is added dropwise to a stirred solution of copper(I)chloride (19.4 g, 0.1956 mmol) in water (200 ml) over 45 minutes and the maximum temperature is kept at 21° C. After 70 minutes at room temperature, the reaction mixture is poured slowly into stirring water and extracted with EtOAc. The combined organic layers are stirred with saturated sodium bicarbonate solution. The organic layer is separated, is washed with water, brine, dried over MgSO₄. After filtration the solvent is evaporated under reduced pressure to give a crude product which is purified by flash chromatography (gradient from isohexane to 47:3 isohexane:EtOAc) to give the titled compound as a white solid.

### b) (3-Chloro-5-nitro-phenyl)-methanol

A solution of 3-chloro-5-nitrobenzoic acid methyl ester (19.0 g, 0.08 mol) in dry toluene (200 ml) is flushed with argon. The colorless solution is cooled to −78° C. and treated with a solution of 1.5M DIBAL (129.2 ml, 0.19 mol) in toluene over 1 hour, maintaining the reaction temperature below −75° C. The reaction mixture is stirred at below −78° C. for 1 hour then slowly allowed to warm to 10° C. The reaction mixture is cooled over an ice-bath and quenched by dropwise addition of 1M HCl (100 ml). The reaction mixture is diluted with water and extracted with EtOAc. The combined organic layers are washed with water, brine, dried over MgSO₄. After filtration, the solvent is evaporated under reduced pressure to give the crude titled compound as a yellow solid.

### c) 3-Chloro-5-nitro-benzaldehyde

To a solution of oxalyl chloride (14.42 ml, 0.167 mol) in dry DCM (130 ml) at −78° C. is added dropwise dry DMSO (26.4 ml, 0.373 mol) in dry DCM (130 ml) over 45 minutes, maintaining the reaction temperature below −70° C., under nitrogen. The solution is stirred at −78° C. for 2 hours. A solution of (3-chloro-5-nitro-phenyl)-methanol (1.67 g, 8.90 mmol) in dry DCM (5 ml) is added dropwise over 15 minutes. The reaction mixture is stirred at −78° C. for 2 hours. Triethylamine (53.47 ml, 0.38 mol) is added dropwise to the reaction mixture over 15 minutes, at below −70° C. The reaction mixture is left in the cooling-bath and allowed to warm to room temperature slowly, then stirred overnight. The reaction mixture is quenched with water and the organic layer is separated. The aqueous is extracted with DCM, the combined organic layers are washed with water, brine, dried over MgSO₄. After filtration, the solvent is removed under reduced pressure to give the crude titled compound as a red-brown solid.

### d) 3-(3-Chloro-5-nitro-phenyl)-acrylonitrile

To a suspension of sodium hydride (60% dispersion in oil, 3.55 g, 0.089 mol) in dry THF (165 ml) at 0° C. is added under nitrogen and dropwise over 15 minutes a solution of diethyl cyanomethylphosphonate (14.1 ml, 0.089 mol) in THF (65 ml) maintaining the reaction temperature below 10° C. The reaction mixture is stirred at 0° C. for 50 minutes. Then a solution of 3-chloro-5-nitro-benzaldehyde (13.84 g, 0.075 mol) in dry THF (45 ml) is added dropwise over 20 minutes, maintaining the reaction temperature below 10° C. The reaction mixture is stirred at 0° C. for 10 minutes then allowed to warm to room temperature and stirred for 3 hours. The reaction mixture is quenched by dropwise addition of water (45 ml). The solvent is removed under reduced pressure. The crude residue is partitioned between EtOAc and water and the aqueous layer is extracted with EtOAc. The combined organic layers are washed with water, brine, dried over MgSO₄. After filtration the solvent is removed under reduced pressure to give the titled product as a brown solid.

### e) 4-(3-Chloro-5-nitro-phenyl)-1H-pyrrole-3-carbonitrile

To a suspension of sodium hydride (60% dispersion in oil, 3.55 g, 0.089 mol) in dry THF (550 ml) is added at 0° C. and under nitrogen a solution of 3-(3-Chloro-5-nitro-phenyl)-acrylonitrile (15.56 g, 0.075 mol) and TosMIC (17.48 g, 0.089 mol) in THF (275 ml) dropwise over 15 minutes, maintaining the reaction temperature below 5° C. The reaction mixture is allowed to warm to room temperature and stirred overnight. The reaction mixture is quenched by dropwise addition of water (55 ml). The solvent is removed under reduced pressure and the crude residue is partitioned between DCM and water. The suspension is filtered and the organic layer and the aqueous layer separated. The solid is dissolved in EtOAc and washed with water. The combined aqueous layers are extracted with EtOAc. The combined organic layers are washed with water, brine, dried over MgSO₄. After filtration, the solvent is removed under reduced pressure to give the crude product as a brown solid. The crude product is triturated in DCM, the solid filtered and dried under vacuum at 40° C. to give the titled compound as pale brown solid; [M+H]⁺ 458.

### f) [3-(3-Chloro-5-nitro-phenyl)-4-cyano-pyrrol-1-yl]-acetic acid ethyl ester

To an ice-cooled stirring solution of t-BuOK (2.38 g, 21.2 mmol) in dry THF (60 ml) under nitrogen is added a solution of 4-(3-chloro-5-nitro-phenyl)-1H-pyrrole-3-carbonitrile (3.50 g, 14.1 mmol) in dry THF (80 ml) dropwise, over 30 minutes. After 3 hours, a solution of ethyl-2-bromoacetate (1.57 ml, 14.1 mmol) in dry THF (60 ml) is added at 0° C. After the addition, the ice bath is removed and the reaction mixture is stirred at room temperature for 1 hour. The solvent is removed under reduced pressure and the residual is partitioned between EtOAc and water. The aqueous layer is extracted with EtOAc, the organic layers are combined, dried over MgSO₄ and the solvent is removed under reduced pressure to give a brown solid. The crude product is purified by flash chromatography (gradient from isohexane to 1:1 isohexane:EtOAc), to afford the titled compound as a yellow solid; [M+MeCN+ H]⁺ 375.

### b) [3-(3-Amino-5-chloro-phenyl)-4-cyano-pyrrol-1-yl]-acetic acidethyl ester

3-(3-Chloro-5-nitro-phenyl)-4-cyano-pyrrol-1-yl]-acetic acid ethyl ester (2.0 g, 6.0 mmol) in EtOH (100 ml) is treated with SnCl₂, 2H₂O (6.76 g, 30.0 mmol) and the reaction mixture is refluxed for 1 hour. The reaction mixture is allowed to cool to room temperature and poured into ice/water. The pH of the solution is adjusted to pH 7-8 by addition of saturated sodium hydrogen carbonate solution, the resulting emulsion is filtered and extracted with EtOAc. The combined organic layers are washed with brine, dried over MgSO₄. After filtration, the solvent is removed under reduced pressure to give an orange oil and dried under vacuum at 40° C. overnight to afford the titled compound.

### c) [3-(3-Chloro-5-chlorosulfonyl-phenyl)-4-cyano-pyrrol-1-yl]-acetic acid ethyl ester

To a solution of [3-(3-amino-5-chloro-phenyl)-4-cyano-pyrrol-1-yl]-acetic acid ethyl ester (1.85 g, 6.0 mmol) in AcOH (41 ml) and concentrated HCl (16.1 ml) is added at 0° C. and dropwise a solution of sodium nitrite (0.414 g, 6.0 mmol) in water (4.2 ml). After stirring at 0° C. for 1.5 hour, the reaction mixture is added dropwise to a stirred solution of SO₂/AcOH/CuCl₂/H₂O (157 ml) (the preparation of the reagent is described herein) over 30 minutes. The reaction mixture is allowed to warm to room temperature and stirred overnight. The reaction mixture is then poured into ice/water (400 ml) and extracted with EtOAc. The combined organic layers are washed with water followed by brine and dried over MgSO₄. After filtration the solvent is removed under reduced pressure to give a red solid. The crude product is purified by flash chromatography (gradient from isohexane to 1:1 isohexane:EtOAc) to afford the titled compound ([M+H₂O]⁺ 458) and [3-(3-chloro-5-chlorosulfonyl-phenyl)-4-cyano-pyrrol-1-yl]-acetic acid. [3-(3-Chloro-5-chlorosulfonyl-phenyl)-4-cyano-pyrrol-1-yl]-acetic acid is used in the next step.

### d) 3-{3-Chloro-5-[4-(2-fluoro-phenyl)-piperazine-1-sulfonyl]-phenyl}-4-cyano-pyrrol-1-yl)-acetic acid, hydrochloride salt

To PS-DIEA (0.113 mg, 0.414 mmol) in THF (1 ml) is added at 0° C. a solution of [3-(3-chloro-5-chlorosulfonyl-phenyl)-4-cyano-pyrrol-1-yl]-acetic acid (50 mg, 0.139 mmol) in THF (1.5 ml) followed by a solution of 1-(2-fluoro-phenyl)-piperazine (22 μl, 0.139 mmol) in THF (1 ml). After the addition, the ice bath is removed and the reaction mixture is stirred at room temperature for 2 hours. The reaction mixture is filtered and the resin washed with THF. The filtrate is evaporated under reduced pressure to give a pink residue. The crude titled compound is dissolved in H₂O/CH₃CN—HCl until pH 1-2 and purified by reverse phase chromatography (gradient 100% H₂O to 100% MeCN) to afford the titled compound; [M+H]⁺ 503.

### Examples 10 to 16

These examples namely,
{3-[3-Chloro-5-(4-pyridin-4-yl-piperazine-1-sulfonyl)-phenyl]-4-cyano-pyrrol-1-yl}-acetic acid, hydrochloride salt (Example 10),
{3-[3-Chloro-5-(4-pyridin-2-yl-piperazine-1-sulfonyl)-phenyl]-4-cyano-pyrrol-1-yl}acetic acid, hydrochloride salt (Example 11),
{3-[3-(4-Benzyl-piperazine-1-sulfonyl)-5-chloro-phenyl]-4-cyano-pyrrol-1-yl}-acetic acid, hydrochloride salt (Example 12),
{3-[3-(4-Benzyl-piperidine-1-sulfonyl)-5-chloro-phenyl]-4-cyano-pyrrol-1-yl}acetic acid, hydrochloride salt (Example 13),
(3-{3-Chloro-5-[4-(2-chloro-phenyl)-piperazine-1-sulfonyl]phenyl}-4-cyano-pyrrol-1-yl)-acetic acid, hydrochloride salt (Example 14) and
{3-[3-Chloro-5-(4-pyridin-4-ylmethyl-piperazine-1-sulfonyl)-phenyl]-4-cyano-pyrrol-1-yl}-acetic acid, hydrochloride salt (Example 15),
   are prepared by similar processes as that described in Example 9.

1. A compound of formula (I) 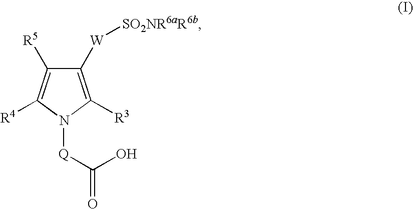
in free or pharmaceutically acceptable salt form, wherein
Q is 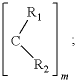
R¹ and R² are, independently, H, halogen, C₁-C₈-alkyl, or together with the carbon atom to which they are attached, form a divalent C₃-C₈-cycloaliphatic group;
R³ and R⁴ are independently selected from H, C₁-C₈-alkyl optionally substituted by C₃-C₁₅ carbocyclic group, or a C₃-C₁₅ carbocyclic group;
R⁵ is selected from H, halogen, C₁-C₈-alkyl, C₁-C₈-haloalkyl, a C₃-C₁₅-carbocyclic group, nitro, cyano, SO₂R^{5a}, SOR^{5b}, SR^{5c}, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, amino(C₁-C₈-alkyl), C₁-C₈-alkylamino, di(C₁-C₈-alkyl)amino, SO₂NR^{5d}R^{5e}, —C(O)NR^{5f}R^{5g}, a C₆-C₁₅-aromatic carbocyclic group, and a 4- to 10-membered heterocyclic group having one or more heteroatoms selected from the group consisting of oxygen, nitrogen and sulphur;
R^{5a}, R^{5b} and R^{5c} are independently selected from C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl and a 4- to 10-membered heterocyclic group having one or more heteroatoms selected from the group consisting of oxygen, nitrogen and sulphur;
R^{5d}, R^{5e}, R^{5f} and R^{5g} are independently H, C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl, a 4- to 10-membered heterocyclic group having one or more heteroatoms selected from the group consisting of oxygen, nitrogen and sulphur, or together with the nitrogen atom to which they are attached, form a C₄-C₁₀-heterocyclic group;
W is selected from C₃-C₁₅-carbocyclic group optionally substituted by halogen, cyano, C₁-C₈-alkyl, or C₁-C₈-haloalkyl, and 4- to 10-membered heterocycle having one or more heteroatoms selected from the group consisting of oxygen, nitrogen and sulphur optionally substituted by halogen, C₁-C₈-alkyl, or C₁-C₈-haloalkyl;
R^{6a} is H or C₁-C₈-alkyl;
R^{6b} is C₁-C₈-alkyl substituted by C₃-C₁₅-carbocyclic group optionally substituted by halogen, C₁-C₈-alkyl, or hydroxyl, or 4- to 10-membered heterocyclic group optionally substituted by halogen, cyano, oxo, hydroxy, carboxy, nitro, or C₁-C₈-alkyl, or
R^{6a} and R^{6b} together with the nitrogen atom to which they are attached, form a 4- to 10 membered heterocyclic group optionally substituted by 4- to 10-membered heterocyclic group, a C₃-C₁₅ carbocyclic group optionally substituted by halogen, C₁-C₈-alkyl or hydroxy, or a C₁-C₈-alkyl optionally substituted by 4- to 10-membered heterocyclic group, or a C₃-C₁₅ carbocyclic group optionally substituted by halogen, C₁-C₈-alkyl or hydroxy;
where each C₃-C₁₅-carbocyclic group, unless otherwise specified, can be optionally substituted by at least one halo, cyano, amino, nitro, carboxy, C₁-C₈-alkyl, C₁-C₈-haloalkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₀-carbocyclic group and a 4- to 10-membered heterocyclic group having at least one ring heteroatom selected from nitrogen, oxygen and sulphur;
and where each 4 to 10-membered heterocyclic group, unless otherwise specified, can be optionally substituted by at least one halo, cyano, oxo, hydroxy, carboxy, nitro, C₁-C₈-alkyl optionally substituted by 4- to 10-membered heterocyclic group, or a C₃-C₁₅ carbocyclic group optionally substituted by halogen, C₁-C₈-alkyl or hydroxy, C₁-C₈-cyanoalkyl, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;
and where each C₈-C₁₅-aromatic carbocyclic group, unless otherwise specified, can be optionally substituted by at least one halo, cyano, amino, nitro, carboxy, C₁-C₈-alkyl, halo-C₁-C₈-alkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₅-carbocyclic group and a 4- to 10-membered heterocyclic group having at least one ring heteroatom selected from nitrogen, oxygen and sulphur; and
m is an integer selected from 1-3.

2. A compound of formula (I) according to claim 1, in free or pharmaceutically acceptable salt form, wherein
R¹ and R² are, independently, H, halogen, or C₁-C₈-alkyl;
R³ and R⁴ are independently selected from H and C₁-C₈-alkyl;
R⁵ is cyano;
R^{6a} is H or C₁-C₈-alkyl;
R^{6b}, C₁-C₈-alkyl substituted by C₃-C₁₅-carbocyclic group optionally substituted by halogen, C₁-C₈-alkyl, or hydroxy or 4- to 10-membered heterocyclic group optionally substituted by halogen, cyano, oxo, hydroxy, carboxy, nitro, or C₁-C₈-alkyl, or
R^{6a} and R^{6b} together with the nitrogen atom to which they are attached, form a 4 to 10-membered heterocyclic group optionally substituted by 4- to 10-membered heterocyclic group, a C₃-C₁₅ carbocyclic group optionally substituted by halogen, C₁-C₈-alkyl or hydroxy), or a C₁-C₈-alkyl optionally substituted by 4- to 10-membered heterocyclic group, or a C₃-C₁₅ carbocyclic group optionally substituted by halogen, C₁-C₈-alkyl or hydroxy;
W is selected from C₃-C₁₅-carbocyclic group optionally substituted by halogen, cyano, C₁-C₈-alkyl, or C₁-C₈-haloalkyl, and 4- to 10-membered heterocycle having one or more heteroatoms selected from the group consisting of oxygen, nitrogen and sulphur optionally substituted by halogen, C₁-C₈-alkyl, or C₁-C₈-haloalkyl; and
m is an integer selected from 1-3.

3. A compound according to claim 1, in free or pharmaceutically acceptable salt form, wherein the compound is of formula (Ia) 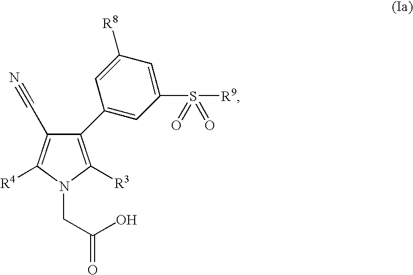
where
R³ and R⁴ are independently selected from H and C₁-C₈-alkyl;
R⁸ is selected from halogen and C₁-C₈-haloalkyl;
R⁹ is selected from NR^{9a}R^{9b};
R^{9a} is H or C₁-C₈-alkyl; and
R^{9b}, C₁-C₈-alkyl substituted by C₃-C₁₅ carbocyclic group or 4- to 10-membered heterocyclic group optionally substituted by C₁-C₈-alkyl, or
R^{9a} and R^{9b} together with the nitrogen atom to which they are attached, form a 4- to 10-membered heterocyclic group optionally substituted by 4- to 1 membered heterocyclic group, a C₃-C₁₅ carbocyclic group optionally substituted by halogen, C₁-C₈-alkyl or hydroxy, or a C₁-C₈-alkyl optionally substituted by 4- to 10-membered heterocyclic group, or a C₃-C₁₅ carbocyclic group optionally substituted by halogen, C₁-C₈-alkyl or hydroxy.

4. A compound according to claim 3, in free or pharmaceutically acceptable salt form, wherein
R³ and R⁴ are H;
R⁸ is selected from Cl and CF₃; and
R⁹ is selected from

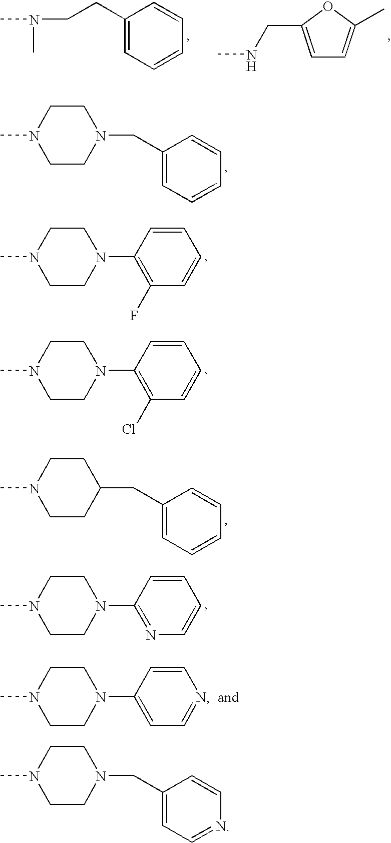

5. A compound according to claim 1, wherein said compound is selected from
{3-[3-(4-Benzyl-piperidine-1-sulfonyl)-5-trifluoromethyl-phenyl]-4-cyano-pyrrol-1-yl}-acetic acid;
{3-Cyano-4-[3-(4-pyridin-2-yl-piperazine-1-sulfonyl)-5-trifluoromethyl-phenyl]-pyrrol-1-yl}-acetic acid, sodium salt;
{3-Cyano-4-[3-(4-pyridin-4-ylmethyl-piperazine-1-sulfonyl)-5-trifluoromethyl-phenyl]-pyrrol-1-yl}-acetic acid, sodium salt;
(3-{3-[4-(2-Chloro-phenyl)-piperazine-1-sulfonyl]-5-trifluoromethyl-phenyl}-4-cyano-pyrrol-1-yl)-acetic acid, sodium salt;
{3-Cyano-4-[3-(4-pyridin-4-yl-piperazine-1-sulfonyl)-5-trifluoromethyl-phenyl]-pyrrol-1-yl}-acetic acid, sodium salt;
{3-[3-(4-Benzyl-piperazine-1-sulfonyl)-5-trifluoromethyl-phenyl]-4-cyano-pyrrol-1-yl}-acetic acid, sodium salt;
{3-[3-Chloro-5-(methyl-phenethyl-sulfamoyl)-phenyl]-4-cyano-pyrrol-1-yl}-acetic acid;
(3-Cyano-4-{3-[(5-methyl-furan-2-ylmethyl)-sulfamoyl]-5-trifluoromethyl-phenyl}-pyrrol-1-yl)-acetic acid;
(3-{3-Chloro-5-[4-(2-fluoro-phenyl)-piperazine-1-sulfonyl]-phenyl}-4-cyano-pyrrol-1-yl)-acetic acid, sodium salt;
{3-[3-Chloro-5-(4-pyridin-4-yl piperazine-1-sulfonyl)-phenyl]-4-cyano-pyrrol-1-yl}-acetic acid, hydrochloride salt;
{3-[3-Chloro-5-(4-pyridin-2-yl-piperazine-1-sulfonyl)-phenyl]-4-cyano-pyrrol-1-yl}-acetic acid, hydrochloride salt;
{3-[3-(4-Benzyl-piperazine-1-sulfonyl)-5-chloro-phenyl]-4-cyano-pyrrol-1-yl}-acetic acid, hydrochloride salt;
{3-[3-(4-Benzyl-piperidine-1-sulfonyl)-5-chloro-phenyl]-cyano-pyrrol-yl}-acetic acid, hydrochloride salt,
(3-{3-Chloro-5-[4-(2-chloro-phenyl)-piperazine-1-sulfonyl]-phenyl}-4-cyano-pyrrol-1-yl)-acetic acid, hydrochloride salt; and
{3-[3-Chloro-5-(4-pyridin-4-ylmethyl-piperazine-1-sulfonyl)-phenyl]-4-cyano-pyrrol-1-yl}-acetic acid, hydrochloride salt.

6. A compound according to claim 1 for use as a pharmaceutical.

7. Pharmaceutical compositions comprising a compound according to claim 1.

8. The use of a compound according to claim 1 in the manufacture of a medicament for treatment of a disease mediated by the CRTh₂ receptor.

9. The use of a compound according to claim 1 in the manufacture of a medicament for treatment of an inflammatory or allergic condition, particularly an inflammatory or obstructive airways disease.

10. A combination of a compound according to claim 1 with an anti-inflammatory, bronchodilatory, antihistamine or anti-tussive drug substance.

11. A process for the preparation of compounds of formula (I) as defined in claim 1, in free or pharmaceutically acceptable salt form, which comprises the steps of:
(i) cleaving an ester group, —COOR⁷ in a compound of formula (II) 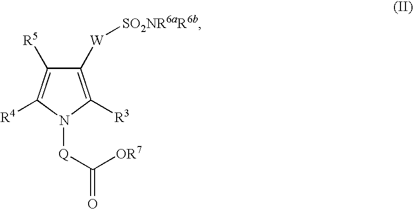
wherein
R⁷ is C₃-C₁₅ carbocyclic group or C₁-C₈-alkyl optionally substituted by a C₃-C₁₅ carbocyclic group; and
everything else as hereinbefore defined; and
(ii) recovering the resultant compound of formula (I), in free or pharmaceutically acceptable salt form.

12. A compound of formula (II) 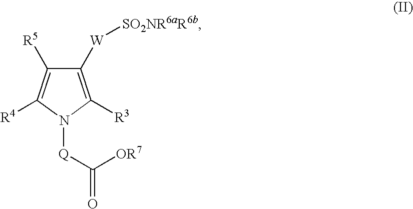
in free or pharmaceutically acceptable salt form, wherein
Q is 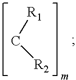
R¹ and R² are, independently, H, halogen, C₁-C₈-alkyl, or together with the carbon atom to which they are attached, form a divalent C₃-C₈-cycloaliphatic group;
R³ and R⁴ are independently selected from H, C₁-C₈-alkyl optionally substituted by C₃-C₁₅ carbocyclic group, or a C₃-C₁₅ carbocyclic group;
R⁵ is selected from H, halogen, C₁-C₈-alkyl, C₁-C₈-haloalkyl, a C₃-C₁₅-carbocyclic group, nitro, cyano, SO₂R^{5a}, SOR^{5b}, SR^{5c}, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, amino(C₁-C₈-alkyl), C₁-C₈-alkylamino, di(C₁-C₈-alkyl)amino, SO₂NR^{5d}R^{5e}, —C(O)NR^{5f}R^{5g}, a C₆-C₁₅-aromatic carbocyclic group, and a 4- to 10-membered heterocyclic group having one or more heteroatoms selected from the group consisting of oxygen, nitrogen and sulphur;
R^{5a}, R^{5b} and R^{5c} are independently selected from C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl, a C₃-C₁₅-carbocyclic group, C₁-C₈-haloalkyl and a 4- to 10-membered heterocyclic group having one or more heteroatoms selected from the group consisting of oxygen, nitrogen and sulphur;
R^{5d}, R^{5e}, R^{5f} and R^{5g} are: independently H, C₁-C₈-alkyl, C₁-C₈-hydroxyalkyl, C₁-C₈-alkylamino(C₁-C₈-alkyl), di(C₁-C₈-alkyl)amino(C₁-C₈-alkyl), C₁-C₈-cyanoalkyl a C₃-C₁₅-carbocyclic group, C₁-C₈-haloalkyl, a 4- to 10-membered heterocyclic group having one or more heteroatoms selected from the group consisting of oxygen, nitrogen and sulphur, or together with the nitrogen atom to which they are attached, form a C₄-C₁₀-heterocyclic group;
W is selected from C₃-C₁₅-carbocyclic group optionally substituted by halogen, cyano, C₁-C₈-alkyl, or C₁-C₈-haloalkyl, and 4- to 10-membered heterocycle having one or more heteroatoms selected from the group consisting of oxygen, nitrogen and sulphur optionally substituted by halogen, C₁-C₈-alkyl, or C₁-C₈-haloalkyl;
R^{6a} is H or C₁-C₈-alkyl;
R^{6b} is C₁-C₈-alkyl substituted by C₃-C₁₅-carbocyclic group optionally substituted by halogen, C₁-C₈-alkyl, or hydroxyl, or 4- to 10-membered heterocyclic group optionally substituted by halogen, cyano, oxo, hydroxy, carboxy, nitro, or C₁-C₈-alkyl, or
R^{6a} and R^{6b} together with the nitrogen atom to which they are attached, form a 4- to 10-membered heterocyclic group optionally substituted by 4- to 10-membered heterocyclic group, a C₃-C₁₅ carbocyclic group optionally substituted by halogen, C₁-C₈-alkyl or hydroxy, or a C₁-C₈-alkyl optionally substituted by 4- to 10-membered heterocyclic group, or a C₃-C₁₅ carbocyclic group optionally substituted by halogen, C₁-C₈-alkyl or hydroxy;
R⁷ is C₃-C₁₅ carbocyclic group or C₁-C₈-alkyl optionally substituted by a C₃-C₁₅ carbocyclic group;
where each C₃-C₁₅-carbocyclic group, unless otherwise specified, can be optionally substituted by at least one halo, cyano, amino, nitro, carboxy, C₁-C₈-alkyl, C₁-C₈-haloalkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₀-carbocyclic group and a 4- to 10-membered heterocyclic group having at least one ring heteroatom selected from nitrogen, oxygen and sulphur;
and where each 4- to 10-membered heterocyclic group, unless otherwise specified, can be optionally substituted by at least one halo, cyano, oxo, hydroxy, carboxy, nitro, C₁-C₈-alkyl optionally substituted by 4- to 10-membered heterocyclic group, or a C₃-C₁₅ carbocyclic group optionally substituted by halogen, C₁-C₈-cyanoalkyl, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and; C₁-C₈-alkoxy optionally substituted by aminocarbonyl;
and where each C₆-C₁₅-aromatic carbocyclic group, unless otherwise specified, can be optionally substituted by at least one halo, cyano, amino, nitro, carboxy, C₁-C₈-alkyl, halo-C₁-C₈-alkyl, C₁-C₈-alkoxy, C₁-C₈-cyanoalkyl, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₅-carbocyclic group and a 4- to 10-membered heterocyclic group having at least one ring heteroatom selected from nitrogen, oxygen and sulphur; and
m is an integer selected from 1-3.
